(19) United States
(12) Patent Application Publication
(10) Pub. No.: US 2006/0252827 A1
(43) Pub. Date: Nov. 9, 2006

(54) Therapeutic use of of acyglycerols and the nitrogen-and sulphur-containing analogues thereof

(76) Inventors: Raphael Darteil, Lille (FR); Kanne Caumont-Bertrand, Frelinghien (FR); Jamila Najib, Santes (FR)

Correspondence Address: NIXON & VANDERHYE, PC, 901 NORTH GLEBE ROAD, 11TH FLOOR, ARLINGTON, VA, 22203, US

(21) Appl. No.: 10/540,482

(22) PCT Filed: Feb. 2, 2004
(86) PCT No.: PCT/FR04/00229

§ 371 Date: Jun. 23, 2005

(30) Foreign Application Priority Data
Jan. 31, 2003 (FR) 0301144

(51) Int. Cl. A61K 31/21; C07C 327/22

(52) U.S. Cl. 514/513; 554/85

(57) ABSTRACT

The invention relates to the use of acylglycerols and the nitrogen- and sulfur-containing analogues thereof in therapy, particularly for the treatment of cerebral ischemia. The invention further relates to methods for preparing said derivatives, novel compounds, in particular acylglycerols, the nitrogen- and sulfur-containing analogues thereof and methods for preparing same.

The invention relates to the use of acylglycerols and the nitrogen- and sulfur-containing analogues thereof in therapy, particularly for the treatment of cerebral ischemia. The invention further relates to methods for preparing said derivatives, novel compounds, in particular acylglycerols, the nitrogen- and sulfur-containing analogues thereof and methods for preparing same.

The compounds according to the invention have advantageous antioxidant and anti-inflammatory pharmacological properties. The invention also describes methods of therapeutic treatment using said compounds and pharmaceutical compositions containing same. In particular, the compounds according to the invention are useful for preventing or treating stroke.

In France, cerebrovascular disease (150,000 new cases annually) is the third leading cause of mortality and the leading cause of disability in adults. Ischemic and hemorrhagic stroke respectively account for 80% and 20% of all cerebrovascular accidents. Cerebral ischemic stroke is an important therapeutic issue that must be addressed in order to reduce the morbidity and mortality of cerebrovascular disease. Progress has been made not only in treating the acute phase of ischemia but also in preventing same. It is therefore important to keep in mind that the identification and management of risk factors are essential in the treatment of this pathology.

Drug-based treatments of cerebral ischemia are based on different strategies. A first strategy comprises preventing the occurrence of cerebral ischemic accidents through prevention of risk factors (hypertension, hypercholesterolemia, diabetes, atrial fibrillation, etc.) or through prevention of thrombosis, in particular with the help of antiplatelet drugs or anticoagulants (Adams 2002).

A second strategy comprises treating the acute phase of ischemia, so as to attenuate its long-term consequences (Lutsep and Clark 2001).

The pathophysiology of cerebral ischemia can be described as follows: the ischemic penumbra, an intermediate zone between the ischemic focus where the neurons are necrotized and the intact nerve tissue, is the site of a pathophysiological cascade which leads over the course of a few days to neuronal death, if reperfusion does not occur or if neuroprotection is insufficient. The first event, which takes place in the first few hours, is a massive release of glutamate which leads to neuron depolarization and cellular oedema. Calcium influx into the cell induces mitochondrial damage leading to the release of free radicals and the induction of enzymes that promote degradation of neuronal membranes. Calcium influx and free radical production in turn activate certain transcription factors, such as NF-κB. Said activation induces inflammatory processes such as induction of endothelial adhesion proteins, polynuclear neutrophil infiltration of the ischemic focus, microgliali activation, induction of enzymes like nitric oxide (NO) synthase type II or cyclooxygenase type II. These inflammatory processes lead to release of NO or prostanoids which are toxic to the cell. Together, these processes result in a phenomenon of apoptosis inducing irreversible lesions (Dirnagl, Iadecola et al. 1999).

The concept of prophylactic neuroprotection is based on experimental data in animal models demonstrating ischemia-resistance. In fact, different procedures applied prior to experimentally induced brain ischemia attenuate the severity of the latter. Various stimuli can induce brain ischemia-resistance: preconditioning (brief ischemia preceding prolonged ischemia); heat stress; administration of a low dose of bacterial lipopolysaccharide (Bordet, Deplanque et al. 2000).

Said stimuli induce resistance mechanisms which activate signals triggering protective mechanisms. Different triggering mechanisms have been identified: cytokines, inflammatory pathways, free radicals, NO, ATP-dependent potassium channels, adenosine. The observed lag time between the onset of early events and ischemia-resistance stems from the need for protein synthesis. Various types of proteins have been shown to induce ischemia-resistance: heat shock proteins, antioxidant enzymes and anti-apoptotic proteins (Nandagopal, Dawson et al. 2001).

Thus there is a real need for compounds capable of preventing the development of risk factors for cerebrovascular accidents such as atherosclerosis, diabetes, obesity, and the like, capable of providing prophylactic neuroprotection but also active neuroprotection in the acute phase of cerebral ischemia.

The PPARs (α,β,γ) belong to the hormone-activated nuclear receptor family. When activated by binding with their ligand, they heterodimerize with Retinoid-X-Receptor (RXR) and bind to “Peroxisome Proliferator Response Elements” (PPREs) located in the promoter sequence of target genes. Binding of PPAR to PPRE thereby induces expression of the target gene (Fruchart, Staels et al. 2001).

The PPARs are distributed in a wide variety of organs, although they all exhibit a certain degree of tissue specificity with the exception of PPARβ the expression of which appears to be ubiquitous. PPARα expression is particularly high in liver and in the intestinal lumen whereas PPARγ is expressed mainly in fat tissue and spleen. The three subtypes (α, β, γ) are expressed in the central nervous system. Cells such as oligodendrocytes and astrocytes more particularly express the PPARα subtype (Kainu, Wikstrom et al. 1994).

The target genes of PPARs control lipid and glucose metabolism. However, recent discoveries suggest that the PPARs participate in other biological processes. PPAR activation by their ligands induces changes in the transcriptional activity of genes which modulate the inflammatory process, antioxidant enzymes, angiogenesis, cell proliferation and differentiation, apoptosis, the activities of iNOS, MMPases and TIMPs (Smith, Dipreta et al. 2001) (Clark 2002).

Free radicals play a role in a very wide range of pathologies including allergy, tumor initiation and promotion, cardiovascular diseases (atherosclerosis, ischemia), genetic and metabolic disorders (diabetes), infectious and degenerative diseases (prion, etc.) and in ophthalmic disorders (Mates, Perez-Gomez et al. 1999).

Reactive oxygen species (ROS) are produced during normal cell functioning. ROS comprise the hydroxyl radical (OH.), superoxide anion (O₂⁻), hydrogen peroxide (H₂O₂) and nitric oxide (NO). Said species are very labile and, due to their high chemical reactivity, constitute a danger to the biological functions of cells. They induce lipid peroxidation, oxidation of certain enzymes and very extensive oxidation of proteins leading to degradation thereof. Protection against lipid peroxidation is a vital process in aerobic organisms, because peroxidation products can cause DNA damage. Thus a deregulation or modification of the equilibrium between the production, processing and elimination of radical species by natural antioxidant defenses leads to the establishment of processes that are deleterious to the cell or organism.

ROS are processed via an antioxidant system that comprises an enzymatic component and a non-enzymatic component. The enzymatic system is composed of several enzymes which have the following characteristics:

Superoxide dismutase (SOD) destroys the superoxide radical by converting it to peroxide. The peroxide in turn is acted upon by another enzyme system. Low levels of SOD are continuously produced by aerobic respiration. Three classes of SOD have been identified in humans, each containing Cu, Zn, Fe, Mn, or Ni as cofactor. The three forms of human SOD are distributed as follows: a cytosolic Cu—Zn SOD, a mitochondrial Mn—SO and an extracellular SOD.
Catalase is very efficient at converting hydrogen peroxide (H₂O₂) to water and O₂. Hydrogen peroxide is enzymatically catabolized in aerobic organisms. Catalase also catalyzes the reduction of a variety of hydroperoxides (ROOH).
Glutathione peroxidase uses selenium as cofactor and catalyzes the reduction of hydroperoxides (ROOH and H₂O₂) by using glutathione, and thereby protects cells against oxidative damage.

Non-enzymatic antioxidant defenses of cells comprise molecules which are synthesized or supplied in the diet.

Antioxidant molecules are present in different cell compartments. Detoxification enzymes for example eliminate free radicals and are essential to cell life. The three most important types of antioxidant compounds are the carotenoids, vitamin C and vitamin E (Gilgun-Sherki, Melamed et al. 2001).

To avoid the phenomenon of apoptosis induced by cerebral ischemia and its resultant effects, the inventors have developed novel compounds capable of preventing the development of the risk factors described earlier and capable of exerting a prophylactic neuroprotective activity, but also of providing active neuroprotection during the acute phase of cerebral ischemia.

The inventors have also shown that the compounds according to the invention concurrently display PPAR activator, antioxidant and anti-inflammatory properties and, as such, said compounds have an important therapeutic or prophylactic potential in cerebral ischemia.

The present invention thus proposes a family of compounds exhibiting advantageous pharmacological properties useful for the preventive or curative treatment of cerebral ischemia. The invention also provides for methods for preparing said derivatives.

The compounds of the invention are represented by general formula (I): 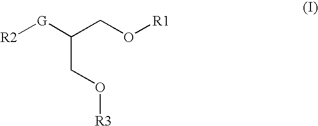
in which:
G represents an oxygen atom, a sulfur atom or a N—R4 group
R4 is a hydrogen atom or a linear or branched alkyl group, saturated or not, optionally substituted, containing from 1 to 5 carbon atoms,
R1, R2 and R3, which are the same or different, represent a hydrogen atom, a CO—R group or a group corresponding to the formula CO—(CH₂)₂ₙ₊₁X—R′, at least one of the groups R1, R2 and R3 is a group corresponding to the formula CO—(CH₂)₂ₙ₊₁—X—R′,
R is a linear or branched alkyl group, saturated or not, optionally substituted, the main chain of which contains from 1 to 25 carbon atoms,
X is a sulfur atom, a selenium atom, a SO group or a SO₂ group,
n is a whole number comprised between 0 and 11,
R′ is a linear or branched alkyl group, saturated or not, optionally substituted, the main chain of which contains from 2 to 23, preferably from 10 to 23, carbon atoms and optionally one or more heterogroups selected in the group consisting of an oxygen atom, a sulfur atom, a selenium atom, a SO group and a SO₂ group.

In the compounds represented by general formula (I) according to the invention, the R group or groups, which are the same or different, preferably represent a linear or branched alkyl group, saturated or unsaturated; substituted or not, the main chain of which contains from 1 to 20 carbon atoms, even more preferably from 7 to 17 carbon atoms, even more preferably from 14 to 17 carbon atoms. In the compounds represented by general formula (I) according to the invention, the R group or groups, which are the same or different, can also represent a lower alkyl group containing from 1 to 6 carbon atoms, such as in particular the methyl, ethyl, propyl, isopropyl, butyl, isobutyl, pentyl or hexyl group.

According to a particular aspect of the invention, the compounds represented by formula (I) are characterized in that one or two of the substituents R1, R2 and R3 is a COCH₃ group.

In the compounds represented by general formula (I) according to the invention, the R′ group or groups, which are the same or different, preferably represent a linear or branched alkyl group, saturated or unsaturated, substituted or not, the main chain of which contains from 12 to 23 carbon atoms, even more preferably from 13 to 20 carbon atoms. Advantageously, R′ represents a linear or branched alkyl group, saturated or unsaturated, substituted or not, the main chain of which contains from 14 to 17 carbon atoms, even more advantageously 14 carbon atoms.

Specific examples of saturated long chain alkyl groups for R or R′ are in particular the groups C₇H₁₅, C₁₀H₂₁, C₁₁H₂₃, C₁₂H₂₅, C₁₃H₂₇, C₁₄H₂₉, C₁₅H₃₁, C₁₆H₃₃, C₁₇H₃₅. Specific examples of unsaturated long chain alkyl groups R or R′ are in particular the groups C₁₄H₂₅, C₁₄H₂₇, C₁₅H₂₉, C₁₇H₂₉, C₁₇H₃₁, C₁₇H₃₃, C₁₉H₂₉, C₁₉H₃₁, C₂₁H₃₁, C₂₁H₃₅, C₂₁H₃₇, C₂₁H₃₉, C₂₃H₄₅, or the alkyl chains of eicosapentanoic (EPA) C_{20:5} (5, 8, 11, 14, 17) and docosahexanoic (DHA) C_{22:6} (4, 7, 10, 13, 16, 19) acids.

Examples of branched long chain alkyl groups are in particular the groups (CH₂)_{n′}—CH(CH₃)C₂H₅, (CH═C(CH₃)—(CH₂)₂)_{n″}—CH═C(CH₃)₂ or (CH₂)₂ₓ₊₁—C(CH₃)₂—(CH₂)_{n′″}—CH₃ (x being a whole number equal to or comprised between 1 and 11, n′ being a whole number equal to or comprised between 1 and 22, n″ being a whole number equal to or comprised between 1 and 5, n′″ being a whole number equal to or comprised between 0 and 22 and (2x+n′″) being less than or equal to 22, preferably less than or equal to 20).

The alkyl groups R or R′ may also contain a cyclic group. Examples of cyclic groups are in particular cyclopropyl, cyclobutyl, cyclopentyl and cyclohexyl.

As indicated earlier, the alkyl groups R or R′ may optionally be substituted by one or more substituents, which are the same or different. The substituents are preferably selected in the group consisting of a halogen atom (iodine, chlorine, fluorine, bromine) and a —OH, ═O, —NO₂, —NH₂, —CN, —CH₂—OH, —O—CH₃, —CH₂OCH₃, CF₃ and COOZ group (Z being a hydrogen atom or an alkyl group, preferably containing from 1 to 6 carbon atoms).

The invention also concerns the optical and geometrical isomers of said compounds, the racemates, salts, hydrates thereof and the mixtures thereof.

Compounds represented by formula (Ia) are compounds corresponding to formula (I) according to the invention in which a single one of the groups R1, R2 or R3 represents a hydrogen atom.

Compounds represented by formula (Ib) are compounds corresponding to formula (I) according to the invention in which two of the groups R1, R2 or R3 represent a hydrogen atom.

According to a particular aspect of the invention, R1 and R3, which are the same or different, represent a hydrogen atom or, more particularly, a CO—R group.

The invention also encompasses the prodrugs of the compounds represented by formula (I) which, after administration to a subject, are converted to compounds represented by formula (I) and/or metabolites of compounds represented by formula (I) which display therapeutic activities similar to compounds represented by formula (I).

The invention also concerns the use of a compound represented by formula (I) for preparing a pharmaceutical composition for treating a cerebrovascular pathology, such as cerebral ischemia or cerebral hemorrhagic stroke.

The invention also deals with a pharmaceutical composition comprising, in a pharmaceutically acceptable support, a compound represented by general formula (I) such as defined hereinabove, possibly in association with another active therapeutic agent. In particular, said composition is intended for the treatment of a cerebrovascular pathology, such as cerebral ischemia or cerebral hemorrhagic stroke.

In the compounds represented by general formula (I) according to the invention, in the group CO—(CH₂)₂ₙ₊₁—X—R′, X preferably represents a sulfur or selenium atom and advantageously a sulfur atom.

Moreover, in the group CO—(CH₂)₂ₙ₊₁—X—R′, n is preferably comprised between 0 and 3, more specifically comprised between 0 and 2 and in particular is equal to 0.

In the compounds represented by general formula (I) according to the invention, R′ may contain one or more heterogroups, preferably 0, 1 or 2, more preferably 0 or 1, selected in the group consisting of an oxygen atom, a sulfur atom, a selenium atom, a SO group or a SO₂ group.

A specific example of a CO—(CH₂)₂ₙ₊₁—X—R′ group according to the invention is the group CO—CH₂—S—C₁₄H₂₉.

In this regard, the inventors have developed novel compounds represented by formula (I) containing a CO—CH₂—S—C₁₄H₂₉ group. Thus, the invention has as object the compounds represented by formula (I) selected from among:
1,3-ditetradecylthioacetyl-2-palmitoylglycerol;
1,3-diacetyl-2-tetradecylthioacetylglycerol;
1,3-dioctanoyl-2-tetradecylthioacetylglycerol;
1,3-diundecanoyl-2-tetradecylthioacetylglycerol; and
1,3-ditetradecylthioacetoxy-2-(2-tetradecylthio)methylcarbonylthio-propane.

Other preferred compounds in the spirit of the invention are compounds represented by general formula (I) hereinabove in which at least one of the groups R1, R2 and R3 represents a CO—(CH₂)₂ₙ₊₁—X—R′ group in which X represents a sulfur or selenium atom, preferably a sulfur atom and/or R′ is a saturated and linear alkyl group containing from 13 to 17 carbon atoms, preferably from 14 to 16, even more preferably 14 carbon atoms.

In this regard, specific compounds according to the invention are those in which R2 is a group having the formula CO—(CH₂)₂ₙ₊₁—X—R′, in which X represents a sulfur or selenium atom, preferably a sulfur atom and/or R′ is a group such as defined hereinabove.

Other particular compounds of the invention are compounds represented by general formula (I) in which the group G advantageously represents an oxygen atom or a N—R4 group, preferably an oxygen atom. Moreover, when G is N—R4, R4 preferably represents a hydrogen atom or a methyl group. In said compounds, R2 advantageously represents a CO—(CH₂)₂ₙ₊₁—X—R′ group such as defined hereinabove.

According to another aspect, particular compounds according to the invention are compounds represented by general formula (I) in which the group G represents a sulfur atom.

Other particular compounds of the invention are those in which two of the groups R1, R2 and R3, which are the same or different, are CO—(CH₂)₂ₙ₊₁—X—R′ groups such as defined hereinabove, in which X represents a sulfur or selenium atom, preferably a sulfur atom.

Other preferred compounds of the invention are compounds represented by general formula (I) in which R1, R2 and R3, which are the same or different, preferably the same, represent a CO—(CH₂)₂ₙ₊₁—X—R′ group such as defined hereinabove, in which X represents a sulfur or selenium atom and preferably a sulfur atom and/or R′ represents a saturated and linear alkyl group containing from 13 to 17 carbon atoms, preferably from 14 to 17, even more preferably 14 carbon atoms, in which n is preferably comprised between 0 and 3, and in particular is equal to 0. More specifically, other preferred compounds are compounds represented by general formula (I) in which R1, R2 and R3 represent CO—CH₂—S—C₁₄H₂₉ groups.

Examples of preferred compounds according to the invention are given in FIGS. 1A and 1B.

Another object of the invention relates to any pharmaceutical composition comprising in a pharmaceutically acceptable support at least one compound represented by formula (I) such as described hereinabove, and in particular at least one compound having formula (I) selected from among:
1,3-ditetradecylthioacetyl-2-palmitoylglycerol;
1,3-diacetyl-2-tetradecylthioacetylglycerol;
1,3-dioctanoyl-2-tetradecylthioacetylglycerol;
1,3-diundecanoyl-2-tetradecylthioacetylglycerol; and
1,3-ditetradecylthioacetoxy-2-(2-tetradecylthio)methylcarbonylthio-propane.

Advantageously, it is a pharmaceutical composition for the treatment or prophylaxis of cerebrovascular pathologies and more particularly cerebral ischemia or cerebrovascular accidents. In fact, it was found surprisingly that compounds represented by formula (I) concurrently display PPAR activator, antioxidant and anti-inflammatory properties and exhibit prophylactic and curative neuroprotective activity in cerebral ischemia.

The invention also concerns the use of a compound such as defined hereinabove for preparing a pharmaceutical composition intended for implementing a method of treatment or prophylaxis in humans or animals.

The invention further concerns a method for treating cerebrovascular pathologies and more particularly cerebral ischemia, comprising administering to a subject, in particular human, an effective dose of a compound represented by formula (I) or of a pharmaceutical composition such as defined hereinabove.

Avantagously, the compounds represented by formula (I) which are used are such as defined hereinabove.

The pharmaceutical compositions according to the invention advantageously comprise one or more pharmaceutically acceptible excipients or vehicles. Examples include pharmaceutically compatible saline, physiologic, isotonic, buffered solutions and the like, known to those skilled in the art. The compositions may contain one or more agents or vehicles selected from among dispersives, solubilizers, stabilizers, surfactants, preservatives, and the like. Agents or vehicles that may be used in the formulations (liquid and/or injectable and/or solid) comprise in particular methylcellulose, hydroxymethylcellulose, carboxymethylcellulose, polysorbate 80, mannitol, gelatin, lactose, vegetable oils, and the like. The compositions may be formulated as injectable suspensions, gels, oils, tablets, suppositories, powders, gelatin capsules, capsules, aerosols, and the like, possibly by means of pharmaceutical forms or devices allowing sustained and/or delayed release. For this type of formulation, an agent such as cellulose, carbonates or starches is advantageously used.

The compounds or compositions of the invention may be administered in different ways and in different forms. For instance, they may be administered systemically, by the oral route, parentally, by inhalation or by injection, such as for example by the intravenous, intramuscular, subcutaneous, transdermal, intra-arterial route, etc. For injections, the compounds are generally prepared in the form of liquid suspensions, which may be injected through syringes or by infusion, for instance. In this respect, the compounds are generally dissolved in pharmaceutically compatible saline, physiologic, isotonic, buffered solutions and the like, known to those skilled in the art. For instance, the compositions may contain one or more agents or vehicles selected from among dispersives, solubilizers, emulsifiers, stabilizers, surfactants, preservatives, buffers, and the like. Agents or vehicles that may be used in the liquid and/or injectable formulations comprise in particular methylcellulose, hydroxymethylcellulose, carboxymethylcellulose, polysorbate 80, mannitol, gelatin, lactose, vegetable oils, liposomes, and the like.

The compositions may thus be administered in the form of gels, oils, tablets, suppositories, powders, gelatin capsules, capsules, aerosols, and the like, possibly by means of pharmaceutical forms or devices allowing sustained and/or delayed release. For this type of formulation, an agent such as cellulose, carbonates or starches is advantageously used.

The compounds may be administered orally in which case the agents or vehicles used are preferably selected in the group consisting of water, gelatin, gums, lactose, starch, magnesium stearate, talc, an oil, polyalkylene glycol, and the like.

For parenteral administration, the compounds are preferably administered in the form of solutions, suspensions or emulsions in particular with water, oil or polyalkylene glycols to which, in addition to preservatives, stabilizers, emulsifiers, etc., it is also possible to add salts to adjust osmotic pressure, buffers, and the like.

It is understood that the injection rate and/or injected dose may be adapted by those skilled in the art according to the patient, the pathology, the mode of administration, etc. Typically, the compounds are administered at doses ranging from 1 μg to 2 g per dose, preferably from 0.1 mg to 1 g per dose. The doses may be administered once a day or several times a day, as the case may be. Moreover, the compositions of the invention may also comprise other active substances or agents.

The invention also concerns methods for preparing the hereinabove compounds represented by formula (I).

The compounds of the invention may be prepared from commercially available products, employing a combination of chemical reactions known to those skilled in the art. The invention also concerns methods for preparing compounds such as defined hereinabove.

According to a first method of the invention, compounds represented by formula (I) in which G is an oxygen or sulfur atom, R1, R2 and R3, which are the same or different, represent a CO—R group or a CO—(CH₂)₂ₙ₊₁—X—R′ group, are obtained from a compound having formula (I) in which G is respectively an oxygen or sulfur atom, R2 is a hydrogen atom and R1 and R3, which are the same or different, represent a CO—R or CO—(CH₂)₂ₙ₊₁—X—R′ group, and a compound having the formula A^{o}-CO-A in which A is a reactive group selected for example in the group consisting of OH, Cl, O—CO-A^{o} and OR″, R″ being an alkyl group, and A^{o} is the R group or (CH₂)₂ₙ₊₁—X—R′ group, possibly in the presence of coupling agents or activators known to those skilled in the art.

Compounds represented by formula (I) according to the invention in which G is an oxygen atom, R2 is a hydrogen atom and R1 and R3, which are the same or different, represent a CO—R or CO—(CH₂)₂ₙ₊₁—X—R′ group, may be obtained in different ways.

In a first embodiment, a glycerol molecule is reacted with a compound having the formula A^{o}-CO-A1 in which A1 is a reactive group selected for example in the group consisting of OH, Cl and OR″, R″ being an alkyl group, and A^{o} is the R group or the (CH₂)₂ₙ₊₁—X—R′ group, possibly in the presence of coupling agents or activators known to those skilled in the art. Said reaction enables the synthesis of so-called symmetrical compounds, in which R1 and R3 have the same meaning. Said reaction may be carried out by adapting the protocols described for example in (Feuge, Gros et al. 1953), (Gangadhar, Subbarao et al. 1989), (Han, Cho et al. 1999) or (Robinson 1960).

Compounds represented by formula (I) according to the invention in which G is an oxygen atom, R2 is a hydrogen atom and R1 and R3, which are the same or different, represent a CO—R or CO—(CH₂)₂ₙ₊₁—X—R′ group, may also be obtained from a compound having formula (I) according to the invention in which G is an oxygen atom, R2 and R3 represent a hydrogen atom and R1 is a CO—R or CO—(CH₂)₂ₙ₊₁—X—R′ group (this particular form of formula (I) compounds being named compounds represented by formula IV), and a compound having the formula A^{o}-CO-A2 in which A2 is a reactive group selected for example in the group consisting of OH and Cl, and A^{o} is the R group or the (CH₂)₂ₙ₊₁—X—R′ group, possibly in the presence of coupling agents or activators known to those skilled in the art. Said reaction is advantageously carried out according to the protocol described for example in (Daubert, Spiegl et al. 1943), (Feuge and Lovegren 1956), (Katoch, Trivedi et al. 1999), (Strawn, Martell et al. 1989) or (Strawn, Martell et al. 1989).

Compounds corresponding to formula IV described hereinabove may be prepared by a method comprising:
a) reacting a compound represented by general formula (II) 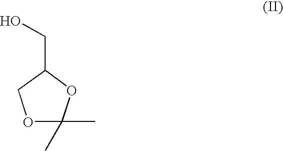
with a compound having the formula A^{o}-CO-A2 in which A2 is a reactive group selected for example in the group consisting of OH and Cl, and A^{o} is the R group or the (CH₂)₂ₙ₊₁—X—R′ group, possibly in the presence of coupling agents or activators known to those skilled in the art, to give a compound represented by general formula (III) 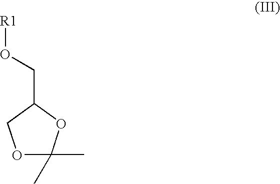
in which R1 represents a CO—R or CO—(CH₂)₂ₙ₊₁—X—R′ group; and
b) deprotecting the compound (III) by an acid (acetic acid, trifluoroacetic acid, boric acid, sulfuric acid, etc.) to give a compound corresponding to general formula (IV) as defined hereinabove.

According to another particular method of the invention, compounds represented by formula (I) in which G is an oxygen atom, R3 is a hydrogen atom and R1 and R2, which are the same or different, represent a CO—R or CO—(CH₂)₂ₙ₊₁—X—R′ group, may be obtained from a compound having formula (I) according to the invention in which G is an oxygen atom, R2 and R3 represent a hydrogen atom and R1 is a CO—R or CO—(CH₂)₂ₙ₊₁—X—R′ group (compounds IV), according to the following steps:
a) reacting compound (IV) with a compound PxE in which Px is a protecting group; and E is a reactive group selected for example in the group consisting of OH and a halogen, to give a compound having general formula (V) in which R1 is a CO—R or CO—(CH₂)₂ₙ₊₁—X—R′ group. Advantageously, the reaction may be carried out by adapting the method described by Gaffney and Reese (1997) in which PxE can represent the compound 9-phenylxanthene-9-ol or 9-chloro-9-phenylxanthene 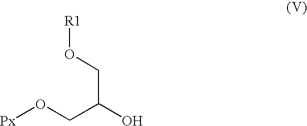
b) reacting the compound having formula (V) with a compound having the formula A^{o}-CO-A2 in which A2 is a reactive group selected for example in the group consisting of OH and Cl, and A^{o} is the R group or the (CH₂)₂ₙ₊₁—X—R′ group, possibly in the presence of coupling agents or activators known to those skilled in the art, to give a compound corresponding to general formula (VI), in which R1 and R2, which are the same or different, represent a CO—R or CO—(CH₂)₂ₙ₊₁—X—R′ group and Px is a protecting group 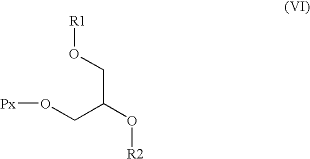
c) deprotecting the compound (VI), in conventional conditions known to those skilled in the art to give a compound represented by general formula (I) in which G is an oxygen atom, R3 is a hydrogen atom and R1 and R2, which are the same or different, represent a CO—R or CO—(CH₂)₂ₙ₊₁—X—R′ group.

According to another specific inventive method, the compounds represented by general formula (I) in which G is an oxygen atom, R1 and R3 represent a hydrogen atom and R2 represents a CO—R or CO—(CH₂)₂ₙ₊₁—X—R′ group, are obtained by a method comprising:
a) reacting a compound represented by formula (VII) 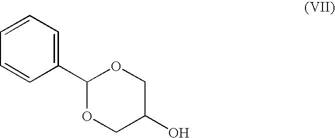
   with a compound having the formula A^{o}—CO-A2 in which A2 is a reactive group selected for example in the group consisting of OH and Cl, and A^{o} is the R group or the (CH₂)₂ₙ₊₁—X—R′ group, possibly in the presence of coupling agents or activators known to those skilled in the art, to give a compound represented by general formula (VIII) 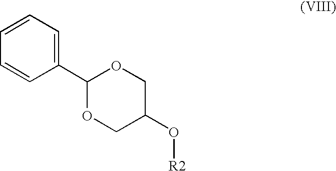
   in which R2 represents a CO—R or CO—(CH₂)₂ₙ₊₁—X—R′ group; and
b) deprotecting the compound represented by formula (VIII) in acidic medium or by catalytic hydrogenation to give a compound having general formula (I) in which G is an oxygen atom, R1 and R3 represent a hydrogen atom and R2 represents a CO—R or CO—(CH₂)₂ₙ₊₁—X—R′ group.

In an advantageous manner, the hereinabove steps may be carried out according to the protocols described in (Bodai, Novak et al. 1999), (Paris, Garmaise et al. 1980), (Scriba 1993) or (Seltzman, Fleming et al. 2000).

Compounds represented by formula (I) according to the invention in which G is a sulfur atom, R2 is a hydrogen atom and R1 and R3, which are the same or different, represent a CO—R or CO—(CH₂)₂ₙ₊₁—X—R′ group, may be obtained from a compound represented by formula (IX) by the following method: 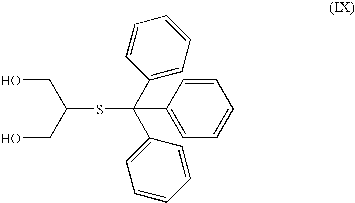
a) reacting the compound (IX) with a first compound having the formula A^{o}-CO-A3 in which A3 is a reactive group selected for example in the group consisting of OH, O—CO-A^{o} and Cl, and A^{o} is the R group or the (CH₂)₂ₙ₊₁—X—R′ group, then with a second compound having the formula A^{o}-CO-A3 in which, independently of the first compound, A3 is a reactive group selected for example in the group consisting of OH, O—CO-A^{o} and Cl, and A^{o} is the R group or the (CH₂)₂ₙ₊₁—X—R′ group, possibly in the presence of coupling agents or activators known to those skilled in the art,
b) deprotecting the thiol group by mercuric acetate.

Said method is advantageously carried out according to the protocol described in (Aveta, Brandt et al. 1986).

Compounds represented by formula (I) according to the invention in which G is a sulfur atom, R2 and R3 are hydrogen atoms and R1 represents a CO—R or CO—(CH₂)₂ₙ₊₁—X—R′ group, may be obtained from a compound represented by formula (IX) by the following method:
a) reacting the compound (IX) with a first compound having the formula A^{o}-CO-A3 in which A3 is a reactive group selected for example in the group consisting of OH, O—CO-A^{o} and Cl, and A^{o} is the R group or the (CH₂)₂ₙ₊₁—X—R′ group in stoichiometric amount, possibly in the presence of coupling agents or activators known to those skilled in the art,
b) deprotecting the thiol group by mercuric acetate.

The compound represented by formula (IX) may be prepared by a method comprising:
a) reacting a dimethyl 2-halogenomalonate with tritylthiol to give a compound represented by formula X 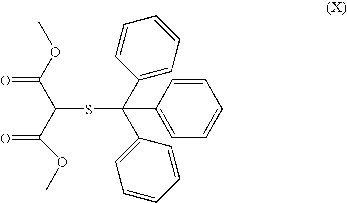
b) reducing the acetate functions with a reducing agent known to those skilled in the art.

Compounds represented by formula (I) according to the invention in which G is an sulfur atom and R1, R2 and R3, which are the same or different, represent a CO—R or CO—(CH₂)₂ₙ₊₁—X—R′ may also be obtained by the following method (see also diagram 1):
a) reacting a compound having formula V with a compound of formula LG-E in which E represents a halogen and LG is a reactive group selected for example in the group consisting of mesyl, tosyl, etc., to give a compound represented by general formula XI in which Px represents a protecting group,
b) reacting a compound having formula XI with a compound of formula Ac-S⁻ B⁺ in which Ac represents a short acyl group, preferably the acetyl group, and B is a counter-ion selected for example in the group consisting of sodium or potassium, preferably potassium to give a compound represented by general formula XIII. Said reaction is advantageously carried out by adapting the protocol described in (Gronowitz, Herslöf et al. 1978),
c) deprotecting the sulfur atom of a compound (XII) in conditions known to those skilled in the art, to give a compound represented by general formula (XIII),
d) reacting a compound represented by general formula (XIII) with a compound having the formula A^{o}-CO-A2 in which A2 is a reactive group selected for example in the group consisting of OH and Cl, and A^{o} is the R group or the (CH₂)₂ₙ₊₁—X—R′ group, possibly in the presence of coupling agents or activators known to those skilled in the art to obtain a compound represented by general formula (XIV) in which R1 and R2, which are the same or different, represent a CO—R or CO—(CH₂)₂ₙ₊₁-X—R′ group,
e) deprotecting a compound of formula (XIV) in conventional conditions known to those skilled in the art, to obtain a compound represented by formula (I) according to the invention in which (i) G is a sulfur atom, (ii) R3 is a hydrogen atom and (iii) R1 and R2 represent a CO—R or CO—(CH₂)₂ₙ₊₁—X—R′ group, the same or different,
f) obtaining various compounds represented by formula (I) according to the invention, in which G is a sulfur atom, and R1, R2 and R3, which are the same or different, represent a CO—R or CO—(CH₂)₂ₙ₊₁—X—R′ group, by reacting compounds having formula (I) according to the invention in which (i) G is a sulfur atom, (ii) R3 is a hydrogen atom and (iii) R1 and R2 represent a CO—R or CO—(CH₂)₂ₙ₊₁—X—R′ group, the same or different, in particular obtained in step e) hereinabove, with a compound having the formula A^{o}-CO-A2 in which A2 is a reactive group selected for example in the group consisting of OH and Cl, and A^{o} is the R group or the (CH₂)₂n+₁—X—R′ group, possibly in the presence of coupling agents or activators known to those skilled in the art.

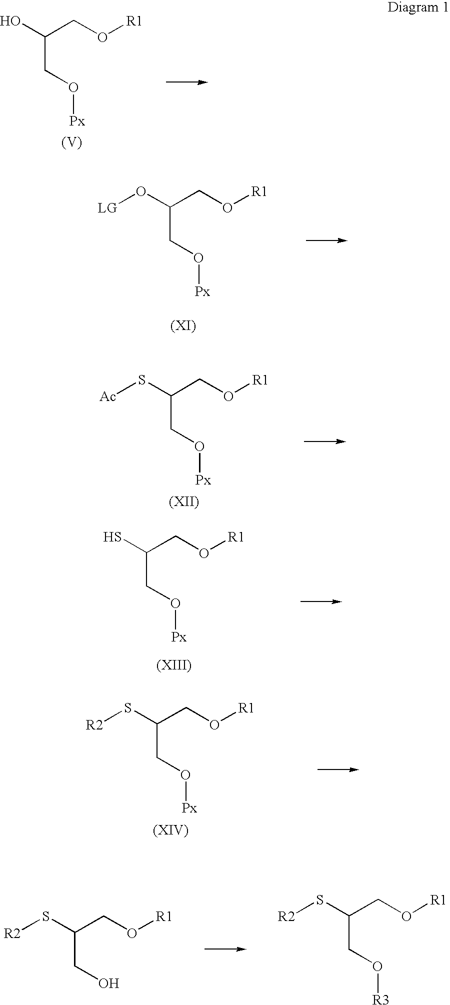

According to another embodiment, compounds represented by formula (I) according to the invention in which G is a sulfur atom, and R1, R2 and R3, which are the same or different, represent a CO—R or CO—(CH₂)₂ₙ₊₁—X—R′ group, may be obtained by the following method:
a) Reacting a compound represented by general formula (I) according to the invention in which (i) G is an oxygen atom (ii) R2 represents a hydrogen atom and (iii) R1 and R3, which are the same or different, represent a CO—R or CO—(CH₂)₂ₙ₊₁—X—R′ group such as defined hereinabove with iodine in the presence of activating agents known to those skilled in the art to obtain a compound having formula (XV) in which R1 and R3, which are the same or different, represent a CO—R or CO—(CH₂)₂ₙ₊₁—X—R′ group. 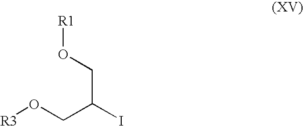
b) reacting a compound having formula (XV) with a thiocarboxylic acid in the presence of coupling agents or activating agents known to those skilled in the art.

Compounds represented by formula (I) in which G is a N—R4 group and in which R1, R2 and R3 which are the same or different, represent a CO—R group or a CO—(CH₂)₂ₙ₊₁—X—R′ group, are obtained from a compound having formula (I) in which G is a N—R4 group, R1 and R3 are hydrogen atoms, R2 is a CO—R group or a CO—(CH₂)₂ₙ₊₁—X—R′ group (compound XVI) according to the following method:

Reacting a compound XVI with a first compound having the formula A^{o}-CO-A2 in which A2 is a reactive group selected for example in the group consisting of OH and Cl, and A^{o} is the R group or the (CH₂)₂ₙ₊₁—X—R′ group, then with a second compound having the formula A^{o}-CO-A2 in which, independently of the first compound, A2 is a reactive group selected for example in the group consisting of OH and Cl, and A^{o} is the R group or the (CH₂)₂ₙ₊₁—X—R′ group, possibly in the presence of coupling agents or activators known to those skilled in the art.

Said method is advantageously carried out according to the protocol described in (Terradas 1993).

Compounds represented by formula (I) according to the invention in which G is a N—R4 group and in which R1 et R2 represent a CO—R or CO—(CH₂)₂ₙ₊₁—X—R′ group, and R3 is a hydrogen atom may be obtained by reacting a compound XI and a compound having the formula A^{o}-CO-A2 in which A2 is a reactive group selected for example in the group consisting of OH and Cl, and A^{o} is the R group or the (CH₂)₂ₙ₊₁—X—R′ group in stoichiometric amount, possibly in the presence of coupling agents or activators known to those skilled in the art.

Compounds represented by formula (I) according to the invention in which G is a NH group, R1 and R3 are hydrogen atoms, R2 is a CO—R group or a CO—(CH₂)₂ₙ₊₁—X—R′ group (compound XVIa) may be obtained in different ways.

In a first method, a molecule of 2-aminopropane-1,3-diol is reacted with a compound having the formula A^{o}-CO-A in which A is a reactive group selected for example in the group consisting of OH, O—CO-A^{o}, OR″ and Cl, and A^{o} is the R group or the (CH₂)₂ₙ₊₁—X—R′ group, possibly in the presence of coupling agents or activators known to those skilled in the art.

Said reaction may be carried out by adapting the protocols described for example in (Shaban 1977), (Kurfürst, Roig et al. 1993), (Harada, Morie et al. 1996), (Khanolkar, Abadji et al. 1996), (Daniher and Bashkin 1998) and (Putnam and Bashkin 2000).

Compounds represented by formula (I) according to the invention in which G is a NH group, R1 and R3 are hydrogen atoms, R2 is a CO—R group or a CO—(CH₂)₂ₙ₊₁—X—R′ group (compound XVIa) may also be obtained by the following method:
a) reacting a compound represented by formula (XVII) with a compound having the formula A^{o}-CO-A in which A is a reactive group selected for example in the group consisting of OH, O—CO-A^{o}, OR″ and Cl, and A^{o} is the R group or the (CH₂)₂ₙ₊₁—X—R′ group, possibly in the presence of coupling agents or activators known to those skilled in the art 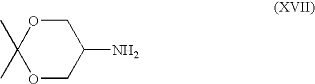
   to give a compound represented by general formula (XVIII) 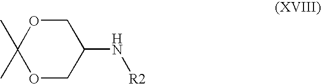
b) deprotecting the compound (XVIII).

Said method is advantageously carried out according to the protocol described in (Harada, Morie et al. 1996).

Compounds represented by formula (I) according to the invention in which G is a N—R4 group in which R4 is not a hydrogen atom, R1 and R3 are hydrogen atoms, R2 is a CO—R group or a CO—(CH₂)₂ₙ₊₁—X—R′ group (compound XVIb) may be obtained according to the following method:
a) reacting a compound having formula (XVII) with a compound having the formula A^{o}-CO-A in which A is a reactive group selected for example in the group consisting of OH, O—CO-A^{o}, OR″ and Cl, and A^{o} is the R group or the (CH₂)₂ₙ₊₁—X—R′ group, possibly in the presence of coupling agents or activators known to those skilled in the art. 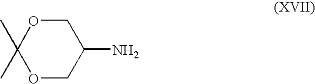
   to give a compound represented by general formula (XVIII) 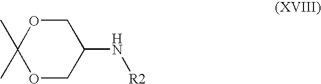
b) reacting the compound (XVIII) with a compound of the type R4-A4 in which A4 is a reactive group selected for example in the group consisting of Cl or Br, in basic medium,
c) deprotecting the compound (XVIII).

The feasibility, realization and other advantages of the invention are further detailed in the following examples, which are given for purposes of illustration and not by way of limitation.

### LEGENDS OF FIGURES

FIG. 1A: Structure of acylglycerols according to the invention (examples 2a, 2c, 4a-r).

FIG. 1B: Structure of particular compounds according to the invention (examples 5a-b, 6-c).

FIG. 2: Evaluation of the antioxidant properties of the inventive compounds on LDL oxidation by copper (Cu).

FIG. 2a: conjugated diene formation over time or lag phase.

FIG. 2b: rate of diene formation.

FIG. 2c: maximum amount of conjugated dienes formed.

FIG. 3: Evaluation of the PPARα agonist properties of the inventive compounds with the Gal4/PPARα transactivation system.

FIG. 4: Evaluation of the neuroprotective effect of the inventive compounds.

FIG. 4a: Prophylactic neuroprotective effect.

FIG. 4b: Prophylactic neuroprotective effect in different regions of the brain.

FIG. 4c: Curative neuroprotective effect (acute phase 24 h).

FIG. 4d: Curative neuroprotective effect in different regions of the brain (acute phase 24 h).

FIG. 4e: Curative neuroprotective effect (acute phase 72 h).

FIG. 4f: Curative neuroprotective effect in different regions of the brain (acute phase 72 h).

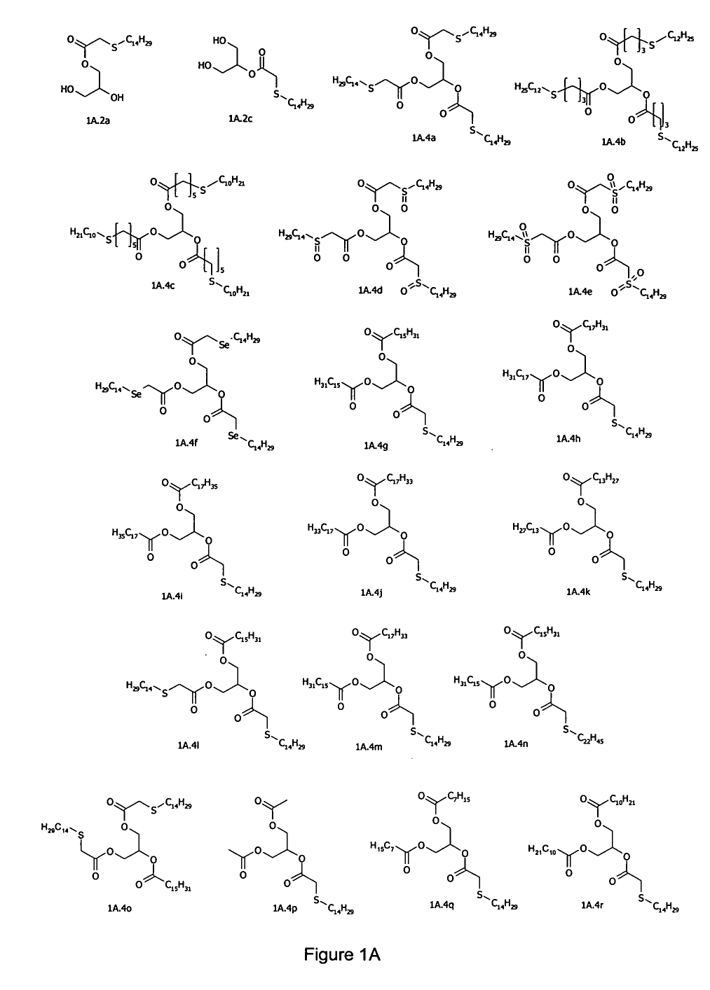

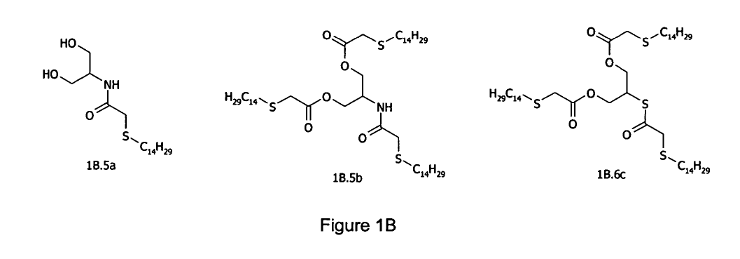

### EXAMPLES

For easier comprehension of the text, the compounds according to the invention used in the examples concerning the measurement and evaluation of activity are abbreviated as follows: “Ex 4g”, for instance, indicates the inventive compound which preparation is described in example 4g.

Thin-layer chromatography (TLC) was carried out on plates coated with MERCK silica gel 60F₂₅₄ 0.2 mm thick. Retention factor is abbreviated Rf.

Column chromatography was carried out on silica gel 60 with a particle size of 40-63 μm (Merck reference 9385-5000).

Melting points (MP) were determined on a Buchi B 540 apparatus by the capillary method. Infrared (IR) spectra were recorded on a Bruker Fourier transformation spectrometer (Vector 22).

Nuclear magnetic resonance (NMR) spectra were recorded on a Bruker AC 300 spectrometer (300 MHz). Each signal was identified by its chemical shift, intensity, multiplicity (noted s for singlet, sl for broad singlet, d for doublet, dd for split doublet, t for triplet, td for split triplet, quint for quintuplet and m for multiplet) and its coupling constant (J).

Mass spectra (MS) were determined on a Perkin Elmer Sciex API 1 (ESI-MS for ElectroSpray Ionization Mass Spectrometry) or on an Applied Biosystems Voyager DE-STR of the MALDI-TOF type (Matrix-Assisted Laser Desorption/Ionization-Time Of Flight).

### Example 1

### Preparation of Fatty Acid Derivatives

### Example 1a

### Preparation of Tetradecylthioacetic Acid

Potassium hydroxide (34.30 g, 0.611 mol), mercaptoacetic acid (20.9 ml, 0.294 mol) and 1-bromotetradecane (50 ml, 0.184 mol) were added in that order to methanol (400 ml). The mixture was stirred overnight at room temperature. A concentrated hydrochloric acid solution (60 ml) dissolved in water (800 ml) was then added to the reaction mixture. The tetradecylthioacetic acid precipitated. The mixture was stirred overnight at room temperature. The precipitate was then filtered, washed five times with water and dried in a dessicator. The product was recrystallized in methanol.

Yield: 94%

Rf (dichloromethane/methanol 9:1): 0.60

MP: 67-68° C.

IR: νCO acid 1726 and 1684 cm⁻¹

NMR (¹H, CDCl₃): 0.84-0.95 (t, 3H, —CH₃, J=6.5 Hz); 1.20-1.45 (multiplet, 22H, —CH₂—); 1.55-1.69 (quint, 2H, —CH₂—CH₂—S—, J=7 Hz); 2.63-2.72 (t, 2H, CH₂—CH₂—S—, J=7 Hz); 3.27 (s, 2H, S—CH₂—COOH)

MS (ESI-MS): M−1=287.

### Example 1b

### Preparation of 4-(dodecylthio)butanoic acid

Dodecanethiol (2.01 g, 10 mmol) and ethyl bromobutyrate (1.971 g, 10 mmol) were stirred at room temperature under an inert atmosphere. Potassium hydroxide (1.36 g, 21 mmol) dissolved in 50 ml of ethanol was added slowly. The reaction mixture was heated under reflux for 3 hours. The ethanol was evaporated under vacuum. The residue was taken up in water and acidified. The precipitate which formed was filtered, washed with water and dried.

Yield: 90%

Rf (dichloromethane/methanol 9:1): 0.46

IR: νCO acid 1689 cm⁻¹

NMR (¹H, CDCl₃): 0.86-0.91 (t, 3H, —CH₃, J=6.2 Hz); 1.25-1.45 (multiplet, 18H, —CH₂—); 1.53-1.63 (quint, 2H, —CH₂—CH₂—S—, J=6.7 Hz); 1.87-2.00 (quint, 2H, —CH₂—S—CH₂—CH₂—CH₂—COOH, J=7.2 Hz); 2.47-2.55 (m, 4H, —CH₂—S—CH₂—CH₂—CH₂—COOH); 2.55-2.62 (t, 2H, —CH₂—S—CH₂—CH₂—CH₂—COOH, J=7.2 Hz)

MS (ESI-MS): M−1=287

### Example 1c

### Preparation of 6-(decylthio)hexanoic acid

Decanethiol (4.57 g, 25 mmol) and 4-bromobutyric acid (5 g, 25 mmol) were stirred at room temperature under an inert atmosphere. Potassium hydroxide dissolved in 50 ml of ethanol was added slowly. The reaction mixture was refluxed for 3 hours. The ethanol was evaporated under vacuum. The residue was taken up in water and acidified. The precipitate which formed was filtered, washed with water and dried.

Yield: 95%

Rf (dichloromethane/methanol 9:1): 0.37

IR: νCO acid 1690 cm⁻¹

NMR (1H, CDCl₃): 0.86-0.91 (t, 3H, —CH₃, J=6.5 Hz); 1.22-1.41 (multiplet, 14H, —CH₂—); 1.42-1.50 (m, 2H, CH₂—S—CH₂—CH₂—CH₂—CH₂—CH₂—COOH); 1.53-1.75 (multiplet, 6H, —CH₂—CH₂—S—CH₂—CH₂—CH₂—CH₂—CH₂—COOH); 2.35-2.42 (t, 2H, —CH₂—S—CH₂—CH₂—CH₂—CH₂—CH₂—COOH, J=7 Hz); 2.48-2.55 (multiplet, 4H, —CH₂—S—CH₂—).

MS (ESI-MS): M−1=287

### Example 1d

### Preparation of Tetradecylselenoacetic Acid

### Preparation of Tetradecyldiselenide

Selenium (1.19 g, 15 mmol) was added under an inert atmosphere to a 1:1 mixture of THF/water (50 ml). After cooling the reaction mix in an ice bath, sodium tetraborohydride (1.325 g, 35 mmol) was added slowly. A second fraction of selenium (1.19, 15 mmol) was added. The reaction mixture was stirred at room temperature for 15 minutes then heated under reflux to dissolve all the reagents. Bromotetradecane (9 ml, 30 mmol) dissolved in 25 ml of tetrahydrofuran was added. The reaction mixture was stirred at room temperature for 3 hours, then extracted with dichloromethane. The organic phases were combined, dried on magnesium sulfate, filtered and evaporated to dryness. The product was used without further purification.

Rf (petroleum ether): 0.77

MP: 43° C.

IR: νCH 2960-2850 cm⁻¹

NMR (¹H, CDCl₃): 0.87-0.93 (t, 6H, —CH₃, J=6.5 Hz); 1.20-1.48 (multiplet, 44H, —CH₂—); 1.62-1.80 (m, 4H, —CH₂—CH₂—Se—); 2.88-2.96 (t, 4H, —CH₂—CH₂—Se—, J=7 Hz).

### Preparation of Tetradecylselenoacetic Acid

In an inert atmosphere, ditetradecyidiselenide (8.5 g, 17 mmol) was dissolved in a mixture of tetrahydrofuran/water (150 ml/50 ml) and cooled in an ice bath. Sodium tetraborohydride (2.9 g, 61 mmol) was added slowly (the solution blanched) followed by bromoacetic acid (8.5 g, 61 mmol) dissolved in a mixture of tetrahydrofuran/water (25 ml/25 ml). The reaction mixture was stirred at room temperature for 6 hours. The reaction mixture was then extracted with ether and the aqueous phase was acidified. The resulting precipitate was filtered, washed several times with water and dried.

Yield: 29%

Rf (dichloromethane/methanol 9:1): 0.60

MP: 68° C.

IR: νCO acid 1719 and 1680 cm⁻¹

NMR (¹H, CDCl₃): 0.85-0.95 (t, 3H, —CH₃, J=6.5 Hz); 1.25-1.48 (multiplet, 22H, —CH₂—); 1.65-1.78 (quint, 2H, —CH₂—CH₂—Se—, J=7 Hz); 2.78-2.84 (t, 2H, CH₂—CH₂—Se—, J=7 Hz); 3.18 (s, 2H, Se—CH₂—COOH)

MS (ESI-MS): M−1=335

### Example 1e

### Preparation of Tetradecylsulfoxyacetic Acid

Tetradecylthioacetic acid (example 1a) (5 g, 17.4 mmol) was dissolved in a mixture of methanol/dichloromethane (160 ml/80 ml). The reaction mixture was stirred and cooled in an ice bath before slowly adding Oxone® (12.8 g, 21 mmol) dissolved in water (160 ml). The reaction mixture was stirred at room temperature for 3 hours. The solvents were evaporated under vacuum. The precipitate which formed in the remaining aqueous phase was drained, washed several times with water and dried.

Yield: 90%

Rf (dichloromethane/methanol 9:1): 0.27

IR: νCO acid 1723 and 1690 cm⁻¹

NMR (¹H, DMSO): 0.80-0.92 (t, 3H, —CH₃, J=6.4 Hz); 1.19-1.50 (multiplet, 22H, —CH₂—); 1.55-1.71 (quint, 2H, —CH₂—CH₂—SO—); 2.70-2.89 (t, 2H, —CH₂—CH₂—SO—CH₂—COOH, J=6.7 Hz); 3.52-3.70 (d, 1H, —CH₂—SO—CH₂—COOH, J=14.5 Hz); 3.80-3.95 (d, 1H, —CH₂—SO—CH₂—COOH, J=14.1 Hz).

MS (ESI-MS): M+1=305; M+23=327 (M+Na⁺); M+39=343 (M+K⁺)

### Example 1f

### Preparation of 6-(decylsulfoxy)hexanoic acid

The product was prepared according to the procedure described hereinabove (example 1e) from 6-(decylthio)hexanoic acid (example 1c).

Yield: 94%.

Rf (dichloromethane/methanol 9:1): 0.18

NMR (¹H, CDCl₃): 0.86-0.91 (t, 3H, —CH₃, J=6.8 Hz); 1.20-1.40 (multiplet, 14H, —CH₂—); 1.40-1.60 (m, 2H, CH₂—SO—CH₂—CH₂—CH₂—CH₂—CH₂—COOH); 1.63-1.95 (multiplet, 6H, —CH₂—CH₂—SO—CH₂—CH₂—CH₂—CH₂—CH₂—COOH); 2.35-2.42 (m, 3H, —CH₂—SO—CH₂—CH₂—CH₂—CH₂—CH₂—COOH and —CH₂—SO—CH₂—CH₂—CH₂—CH₂—CH₂—COOH); 2.60-2.71 (m, 1H, —CH₂—SO—CH₂—CH₂—CH₂—CH₂—CH₂—COOH); 2.75-2.85 (m, 1H, —CH₂—SO—(CH₂)₅—COOH); 2.80-3.01 (m, 1H, —CH₂—SO—(CH₂)₅—COOH).

### Example 1g

### Preparation of Tetradecylsulfonylacetic Acid

Tetradecylthioacetic acid (example 1a) (5 g, 17.4 mmol) was dissolved in a mixture of methanol/dichloromethane (160 ml/80 ml). The reaction mixture was stirred and cooled in an ice bath before slowly adding Oxone® (21.8 g, 35 mmol) dissolved in water (160 ml). The reaction mixture was stirred at room temperature for 3 hours. The solvents were evaporated under vacuum. The precipitate which formed in the remaining aqueous phase was drained, washed several times with water and dried.

Yield: 89%

Rf (dichloromethane/methanol 9:1): 0.21

IR: νCO acid 1701 cm⁻¹

NMR (¹H, DMSO): 0.85-0.96 (t, 3H, —CH₃, J=6 Hz); 1.20-1.40 (multiplet, 20H, —CH₂—); 1.40-1.55 (m, 2H, —CH₂—CH₂—CH₂—SO₂—); 1.80-1.96 (m, 2H, —CH₂—CH₂—SO₂); 3.22-3.34 (t, 2H, —CH₂—CH₂—SO₂—CH₂—COOH, J=8 Hz); 4.01 (s, 2H, —CH₂—SO₂—CH₂—COOH).

MS (ESI-MS): M−1=319

### Example 1h

### Preparation of 6-(decylsulfonyl)hexanoic acid

The product was prepared according to the procedure described hereinabove (example 1g) from 6-(decylthio)hexanoic acid (example 1c).

Yield: 87%

Rf (dichloromethane/methanol 9:1): 0.15

IR: νCO acid 1689 cm⁻¹

NMR (1H, CDCl₃): 0.85-0.96 (t, 3H, —CH₃, J=6.5 Hz); 1.22-1.40 (multiplet, 14H, —CH₂—); 1.40-1.61 (m, 2H, —SO₂—CH₂—CH₂—CH₂—); 1.65-1.95 (multiplet, 6H, —CH₂—CH₂—SO₂—CH₂—CH₂—CH₂—CH₂—CH₂—COOH); 2.35-2.46 (m, 2H, —CH₂—COOH); 2.60-2.84 (m, 2H, —CH₂—SO₂—CH₂—CH₂—CH₂—CH₂—CH₂—COOH); 2.90-3.02 (m, 2H, —CH₂—SO₂—CH₂—CH₂—CH₂—CH₂—CH₂—COOH).

### Example 1i

### Preparation of Docosylthioacetic Acid

The product was obtained according to the procedure described hereinabove (example 1a) from mercaptoacetic acid and bromodocosane.

Yield: 90%

Rf (dichloromethane/methanol 9:1): 0.62

IR: νCO acid 1728 and 1685 cm⁻¹

NMR (¹H, CDCl₃): 0.83-0.94 (t, 3H, —CH₃, J=6.6 Hz); 1.18-1.48 (multiplet, 38H, —CH₂—); 1.55-1.69 (quint, 2H, —CH₂—CH₂—S—, J=7 Hz); 2.63-2.72 (t, 2H, CH₂—CH₂—S—, J=7 Hz); 3.26 (s, 2H, S—CH₂—COOH)

### Example 2

### Preparation of Monoacylglycerols

### Example 2a

### Preparation of 1-tetradecylthioacetylglycerol

### Preparation of 1-tetradecylthioacetyl-2,3-isopropylideneglycerol

In a flask immersed in an ice bath, tetradecylthioacetic acid (example 1a) (4 g, 13.86 mmol) was dissolved in tetrahydrofuran (100 ml) after which EDCl (2.658 g, 13.86 mmol), dimethylaminopyridine (1.694 g, 13.86 mmol) and solketal (1.72 ml, 13.86 mmol) were added in that order. The reaction mixture was stirred at room temperature for 4 days. The solvent was evaporated under vacuum. The residue was taken up in dichloromethane, washed with an aqueous solution of 1 N hydrochloric acid then with an aqueous solution of 10% sodium bicarbonate and lastly with a saturated sodium chloride solution. The organic phase was dried on magnesium sulfate, filtered and evaporated under vacuum. The residual oil was purified by silica gel chromatography (ethyl acetate/cyclohexane 1:9). The product was obtained as a yellow oil.

Yield: 80%

Rf (cyclohexane/ethyl acetate 8:2): 0.65

IR: νCO ester 1736 cm⁻¹

NMR (¹H, CDCl₃): 0.86 (t, 3H, —CH₃, J=7.8 Hz); 1.25 (multiplet, 20H, —CH₂—); 1.33 (s, 3H, CH₃ isopropylidene); 1.37 (s, 3H, CH₃ isopropylidene); 1.59 (m, 4H, OCO—CH₂—S—CH₂—CH₂—CH₂—); 2.62 (t, 2H, —O—CO—CH₂—S—CH₂—, J=7.4 Hz); 3.25 (s, 2H, —O—CO—CH₂—S—CH₂—); 3.75 (m, 1H, —CO—O—CH₂—CH(O)—CH₂(O) (isopropylidene)); 4.08 (m, 2H, —CO—O—CH₂—CH(O)—CH₂(O)— (isopropylidene)); 4.18 (m, 1H, —CO—O—CH₂—CH(O)—CH₂(O)— (isopropylidene); 4.35 (m, 1H, —CO—O—CH₂—CH(O)—CH₂(O)-(isopropylidene)).

### Preparation of 1-tetradecylthioacetylglycerol

1-tetradecylthioacetyl-2,3-isopropylideneglycerol (4.163 g, 10.356 mmol) was dissolved in acetic acid (60 ml) and stirred at room temperature. After 11 days of reaction, the reaction mixture was diluted in water, then extracted with ethyl acetate. The organic phase was washed with a saturated aqueous sodium chloride solution then dried on magnesium sulfate, filtered and the solvent was evaporated. The resulting white powder was recrystallized in heptane.

Yield: 90%

Rf (ethyl acetate/cyclohexane 5:5): 0.30

MP: 63-65° C.

IR: νCO ester 1720 cm⁻¹

NMR (1H, CDCl₃): 0.89 (t, 3H, —CH₃, J=6.6 Hz); 1.28 (multiplet, 20H, —CH₂—); 1.59 (multiplet, 4H, —CH₂—CH₂—CH₂—S—); 2.64 (t, 2H, CH₂—CH₂—S—, J=7.2 Hz); 3.26 (s, 2H, S—CH₂—COO); 3.64 (m, 2H, —COO—CH₂—CHOH—CH₂OH); 3.97 (m, 1H, —COO—CH₂—CHOH—CH₂OH); 4.27 (m, 2H, —COO—CH₂—CHOH—CH₂OH).

MS (MALDI-TOF): M+23=385 (M+Na⁺)

### Example 2b

### Preparation of 1-palmitoylglycerol

This compound was synthesized according to the procedure described hereinabove (example 2a) starting from solketal and palmitic acid.

### 1-palmitoyl-(2,3-isopropylidene)glycerol

Yield: 55%

Rf (dichloromethane): 0.35

MP: 32-33° C.

IR: νCO ester 1733 cm⁻¹

NMR (1H, CDCl₃): 0.89 (t, 3H, —CH₃, J=6.6 Hz); 1.27 (multiplet, 24H, —CH₂—); 1.39 (s, 3H, CH₃ isopropylidene); 1.45 (s, 3H, CH₃ isopropylidene); 1.62 (m, 2H, OCO—CH₂—CH₂—CH₂—); 2.32 (t, 2H, —O—CO—CH₂—CH₂—CH₂—, J=7.4 Hz); 3.75 (dd, 1H, CO—O—CH₂—CH(O)—CH₂(O) (isopropylidene), J=8.3 Hz and J=2.1 Hz); 4.10 (m, 2H, —CO—O—CH₂—CH(O)—CH₂(O)— (isopropylidene)); 4.18 (dd, 1H, —CO—O—CH₂—CH(O)—CH₂(O)— (isopropylidene), J=11.6 Hz and J=4.6 Hz); 4.33 (m, 1H, —CO—O—CH₂—CH(O)—CH₂(O)— (isopropylidene)).

### 1-palmitoylglycerol

Yield: 84%

Rf (ethyl acetate/cyclohexane 5:5): 0.30

MP: 72-74° C.

IR: νCO ester 1730 cm⁻¹

NMR (1H, CDCl₃): 0.89 (t, 3H, —CH₃, J=6.5 Hz); 1.26 (multiplet, 24H, —CH₂—); 1.64 (m, 2H, OCO—CH₂—CH₂—CH₂—); 2.36 (t, 2H, —O—CO—CH₂—CH₂—CH₂—, J=7.4 Hz); 3.60 (dd, 1H, —CO—O—CH₂—CHOH—CH₂OH, J=11.8 Hz and J=6.1 Hz); 3.71 (dd, 1H, —CO—O—CH₂—CHOH—CH₂OH, J=11.8 Hz and J=3.9 Hz); 3.94 (m, 1H, —CO—O—CH₂—CHOH—CH₂OH); 4.19 (m, 2H, —CO—O—CH₂—CHOH—CH₂OH).

### Example 2c

### Preparation of 2-tetradecylthioacetylglycerol

### Preparation of 1,3-benzylideneglycerol

Glycerol (30 g, 0.326 mol), benzaldehyde (34.5 g, 0.326 mol) and p-toluene sulfonic acid (50 mg) were dissolved in 350 ml of toluene and refluxed in a Dean-Stark apparatus for 18 hours. The reaction mixture was dried. The residual product was purified by silica gel chromatography (eluent: cyclohexane/ethyl acetate 8:2 then 7:3) and recrystallized.

Yield: 20%

Rf (ethyl acetate/cyclohexane 5:5): 0.34

IR: νOH 3286 cm⁻¹

NMR (1H, CDCl₃): 3.19 (sl, 1H exchangeable, —OH); 3.64 (sl, 1H, —O—CH₂—CHOH—CH₂O—); 3.99-4.16 (dd, 2H, —O—CHaHb-CHOH—CHaHbO—, J=1.1 Hz and J=10.4 Hz); 4.17-4.23 (dd, 2H, —O—CHaHb-CHOH—CHaHbO—, J=1.6 Hz and J=11.5 Hz); 5.57 (s, 1H, Φ-CH—); 7.34-7.45 (m, 3H, aromatic H); 7.49-7.55 (m, 2H, aromatic H).

### Preparation of 2-tetradecylthioacetyl-1,3-benzylideneglycerol

In a flask immersed in an ice bath, tetradecylthioacetic acid (example 1a) (0.800 g, 2.774 mmol) was dissolved in tetrahydrofuran (75 ml) followed by addition of EDCl (0.532 g, 2.774 mmol), dimethylaminopyridine (0.339 g, 2.774 mmol) and 1,3-benzylideneglycerol (0.5 g, 2.774 mmol) in that order. The mixture was stirred at room temperature for 16 hours. The solvent was evaporated. The residue was taken up in dichloromethane, washed with 1 N hydrochloric acid then with a 10% potassium carbonate solution and lastly with a saturated aqueous sodium chloride solution. The organic phase was dried on magnesium sulfate, filtered and dried. The residue was taken up in petroleum ether. The precipitate which formed was filtered, then purified by silica gel chromatography (eluent:ethyl acetate/cyclohexane 2:8) to produce the desired compound as a white powder.

Yield: 50%

Rf (ethyl acetate/cyclohexane 2:8): 0.53

MP: 51-53° C.

IR: νCO ester 1723 cm⁻¹

NMR (1H, CDCl₃): 0.85-0.96 (t, 3H, CH₃, J=6.8 Hz); 1.19-1.44 (multiplet, 20H, —CH₂); 1.52-1.69 (multiplet, 4H, —CH₂—CH₂—CH₂—S—); 2.62-2.80 (t, 2H, —CH₂—CH₂—CH₂—S—, J=7.2 Hz); 3.34 (s, 2H, —CH₂—S—CH₂—COO—); 4.12-4.29 (dd, 2H, —O—CHaHb-CH(OCO)—CHaHbO—, J=1.7 Hz and J=13.1 Hz); 4.30-4.41 (dd, 2H, —O—CHaHb-CH(OCO)—CHaHbO—, J=1.3 Hz and J=13.1 Hz); 4.75-4.79 (t, 1H, —O—CH₂—CH(OCO)—CH₂O—, J=1.7 Hz); 5.59 (s, 1H, Φ-CH—); 7.35-7.45 (m, 3H, aromatic H); 7.48-7.57 (m, 2H, aromatic H).

### Preparation of 2-tetradecylthioacetylglycerol

2-tetradecylthioacetyl-1,3-benzylideneglycerol (0.576 g, 1.278 mmol) was dissolved in a 50:50 (V/V) mixture of dioxane and triethylborate followed by addition of boric acid (0.317 g, 5.112 mmol). The reaction mixture was heated at 100° C. for 4 hours. Two equivalents of boric acid (0.158 g, 2.556 mmol) were added followed by 2 equivalents after 5.5 hours and 7 hours of reaction. After 24 hours of reaction, the triethylborate was evaporated. The residue was taken up in ethyl acetate and washed with water. The aqueous phase was neutralized with sodium bicarbonate then extracted with dichloromethane. The organic phase was washed with a saturated aqueous sodium chloride solution, dried on magnesium sulfate, filtered and dried. The residue was purified by silica gel chromatography (eluent:ethyl acetate/cyclohexane 5:5).

Yield: 62%

Rf (ethyl acetate/cyclohexane 7:3): 0.51

IR: νCO ester 1739 cm⁻¹

NMR (1H, CDCl₃): 0.82-0.95 (t, 3H, —CH₃, J=6.9 Hz); 1.15-1.35 (multiplet, 22H, —CH₂—); 1.55-1.68 (m, 2H, —CH₂—CH₂—S—); 2.23 (sl, 2H, OH); 2.65 (m, 2H, CH₂—CH₂—S—); 3.26 (s, 2H, S—CH₂—COO); 3.64-3.73 (m, 4H, HOCH₂—CH(OCO—R)—CH₂OH); 3.97 (m, 1H, HOCH₂—CH(OCO—R)—CH₂OH).

### Example 3

### Preparation of 1,3-diacylglycerols

### Example 3a

### Preparation of 1,3-dipalmitoylglycerol

Glycerol (10 g, 0.109 mol, 1 eq), palmitic acid (55.69 g, 0.217 mol, 2 eq), dicyclohexylcarbodiimide (44.77 g, 0.217 mol, 2 eq) and dimethylaminopyridine (26.51 g, 0.217 mol, 2 eq) were dissolved in dichloromethane. The reaction mixture was stirred at room temperature for 48 hours. The dicyclohexylurea which formed was filtered and washed several times with dichloromethane. The filtrate was dried. The residual product was purified by silica gel chromatography (eluent: dichloromethane).

Yield: 45%

Rf (dichloromethane): 0.30

MP: 70-73° C.

IR: νCO ester 1735 and 1716 cm⁻¹

NMR (¹H, CDCl₃): 0.86-91 (t, 6H, —CH₃, J=6.5 Hz); 1.27 (multiplet, 48H, —CH₂—); 1.60-1.65 (quint, 4H, OCOCH₂—CH₂—, J=7.4 Hz); 2.32-2.38 (t, 4H, OCOCH₂—CH₂, J=7.6 Hz); 2.51-2.52 (d, 1H, OH (exchangeable)); 4.06-4.21 (multiplet, 5H, —CH₂—CH—CH₂—)

MS (MALDI-TOF): M+23=591 (M+Na⁺); M+39=607 (M+K⁺)

### Example 3b

### Preparation of 1,3-dilinoleylglycerol

This compound was obtained according to the procedure described hereinabove (example 3a) from glycerol and linoleic acid. The product was obtained as a colorless oil.

Yield: 26%

Rf (dichloromethane): 0.30

IR: νCO ester 1743 and 1719 cm⁻¹

NMR (1H, CDCl₃): 0.83-0.93 (t, 6H, —CH₃, J=6.5 Hz); 1.15-1.44 (multiplet, 28H, —CH₂—); 1.55-1.70 (quint, 4H, OCOCH₂—CH₂—, J=7.4 Hz); 1.90-2.15 (multiplet, 8H, —CH₂—CH═CH—CH₂—CH═CH—CH₂—); 2.30-2.41 (t, 4H, OCOCH₂—CH₂—, J=7.6 Hz); 2.48-2.52 (d, 1H, OH (exchangeable)); 2.70-2.83 (t, 4H, —CH₂—CH═CH—CH₂—CH═CH—CH₂—); 4.05-4.25 (multiplet, 5H, —CHaHb-CH—ChaHb-); 5.25-5.46 (m, 8H, —CH₂—CH═CH—CH₂—CH═CH—CH₂—).

MS (MALDI-TOF): M+23=639 (M+Na⁺); M+39=655 (M+K⁺)

### Example 3c

### Preparation of 1,3-distearylglycerol

This compound was obtained according to the procedure described hereinabove (example 3a) from glycerol and stearic acid. The product was obtained as a white powder.

Yield: 21%

Rf (dichloromethane): 0.30

IR: νCO ester 1735 and 1716 cm⁻¹

NMR (¹H, CDCl₃): 0.83-0.91 (t, 6H, —CH₃, J=6.5 Hz); 1.27 (multiplet, 56H, —CH₂—); 1.59-1.66 (quint, 4H, OCOCH₂—CH₂—, J=7.4 Hz); 2.33-2.38 (t, 4H, OCOCH₂—CH₂—, J=7.5 Hz); 2.45-2.47 (d, 1H, OH (exchangeable), J=4.3 Hz); 4.08-4.23 (multiplet, 5H, —CHaHb-CH—CHaHb-).

MS (MALDI-TOF): M+23=647 (M+Na⁺)

### Example 3d

### Preparation of 1,3-dioleylglycerol

This compound was obtained according to the procedure described hereinabove (example 3a) from glycerol and oleic acid. The product was obtained as a colorless oil.

Yield: 15%

Rf (dichloromethane): 0.23

IR: νCO ester 1743 and 1720 cm⁻¹

NMR (¹H, CDCl₃): 0.89 (t, 6H, —CH₃, J=7.2 Hz); 1.30 (multiplet, 40H, —CH₂—); 1.64 (quint, 4H, OCOCH₂—CH₂—, J=7.4 Hz); 2.02 (multiplet, 8H, —CH₂—CH═CH—CH₂—); 2.36 (t, 4H, OCOCH₂—CH₂—, J=7.2 Hz); 2.45 (d, 1H, OH (exchangeable), J=4.2 Hz); 4.18 (multiplet, 5H, —CHaHb-CH—CHaHb-); 5.35 (m, 4H, —CH₂—CH═CH—CH₂—).

MS (MALDI-TOF): M+23=643 (M+Na⁺)

### Example 3e

### Preparation of 1,3-ditetradecanoylglycerol

This compound was obtained according to the procedure described hereinabove (example 3a) from glycerol and tetradecanoic acid. The product was obtained as a white powder.

Yield: 30%

Rf (dichloromethane): 0.30

IR: νCO ester 1733 and 1707 cm⁻¹

NMR (¹H, CDCl₃): 089 (t, 6H, —CH₃, J=6.5 Hz); 1.26 (multiplet, 40H, —CH₂—); 1.62 (quint, 4H, OCOCH₂—CH₂—, J=7.4 Hz); 2.36 (t, 4H, OCOCH₂—CH₂—, J=7.5 Hz); 2.45 (d, 1H, OH (exchangeable), J=4.3 Hz); 4.15 (multiplet, 5H, —CHaHb-CH—CHaHb-).

### Example 3f

### Preparation de 1,3-ditetradecylthioacetylglycerol

This compound was obtained according to the procedure described hereinabove (example 3a) from glycerol and tetradecylthioacetic acid (example 1a). The product was obtained as a white powder.

Yield: 37%

Rf (dichloromethane): 0.27

MP: 71-73° C.

IR: νCO ester 1704 cm⁻¹

NMR (¹H, CDCl₃): 089 (t, 6H, —CH₃, J=6.3 Hz); 1.27 (multiplet, 44H, —CH₂—); 1.58-1.63 (m, 4H, —OCO—CH₂—S—CH₂—CH₂—); 2.64 (t, 4H, —OCO—CH₂—S—CH₂—CH₂—, J=7.4 Hz); 3.26 (s, 4H, —OCO—CH₂—S—CH₂—); 4.16-4.29 (multiplet, 5H, —CHaHb-CH—CHaHb-).

### Example 3g

### Preparation of 1-oleyl-3-palmitoylglycerol

Glycerol 1-palmitate (example 2b) (5.516 g, 0.017 mol) was dissolved in dichloromethane (500 ml). Dicyclohexylcarbodiimide (5.165 g, 0.025 mol), dimethylaminopyridine (3.058 g, 0.025 mol) and oleic acid (4.714 g, 0.017 mol) were then added. The reaction mixture was stirred at room temperature for 24 hours. The dicyclohexylurea precipitate was filtered, washed with dichloromethane and the filtrate was evaporated under vacuum. The residue obtained was purified by silica gel chromatography (eluent:dichloromethane) to give the desired compound as a white solid.

Yield: 23%

Rf (dichloromethane): 0.24

MP: 30° C.

IR: νCO ester 1731 and 1710 cm⁻¹

NMR (¹H, CDCl₃): 087 (t, 6H, —CH₃, J=6.5 Hz); 1.26 (multiplet, 44H, —CH₂—); 1.62 (quint, 4H, OCOCH₂—CH₂—, J=7.4 Hz); 2.01 (multiplet, 4H, —CH₂—CH═CH—CH₂—); 2.36 (t, 4H, OCOCH₂—CH₂—, J=7.3 Hz); 2.465 (d, 1H, OH (exchangeable), J=4.3 Hz); 4.17 (multiplet, 5H, —CHaHb-CH—CHaHb-); 5.34 (m, 4H, —CH₂—CH═CH—CH₂—).

MS (MALDI-TOF): M+23=617 (M+Na⁺)

### Example 3h

### Preparation of 1,3-diacetylglycerol

Glycerol (30 g, 0.326 mol) was dissolved in dichloromethane (300 ml) followed by addition of pyridine (79 ml, 0.977 mol) and then dropwise addition of acetic anhydride (61.5 ml, 0.651 mol). The reaction mixture was stirred at room temperature for 48 hours. The mixture was taken up in dichloromethane. The organic phase was washed with 1N hydrochloric acid then with 10% sodium bicarbonate then with a saturated aqueous sodium chloride solution, dried on magnesium sulfate, filtered, and evaporated to dryness to provide a colorless oil which was used without further purification.

Yield: 34%

IR: νCO ester 1742 cm⁻¹

### Example 3i

### Preparation of 1,3-dioctanoylglycerol

This compound was obtained according to the procedure described hereinabove (example 3a) from glycerol and octanoic acid. The product was obtained as a colorless oil.

Yield: 10%

Rf (ethyl acetate/cyclohexane 3:7): 0.55

MP <4° C.

IR: νCO ester 1742 and 1719 cm⁻¹

NMR (¹H, CDCl₃): 0.89 (t, 6H, —CH₃, J=6.9 Hz); 1.29 (multiplet, 16H, —CH₂—); 1.62 (multiplet, 4H, OCOCH₂—CH₂—); 2.36 (t, 4H, OCOCH₂—CH₂—, J=7.4 Hz); 2.52 (sl, 1H, OH (exchangeable)); 4.14 (multiplet, 5H, —CH₂—CH—CH₂—)

MS (MALDI-TOF): M+23=591 (M+Na⁺); M+39=607 (M+K⁺)

### Example 3j

### Preparation of 1,3-diundecanoylglycerol

This compound was obtained according to the procedure described hereinabove (example 3a) from glycerol and undecanoic acid. The product was obtained as a white powder.

Yield: 28%

Rf (dichloromethane): 0.20

IR: νCO ester 1730 and 1705 cm⁻¹

NMR (¹H, CDCl₃): 0.89 (t, 6H, —CH₃, J=6.7 Hz); 1.27 (multiplet, 28H, —CH₂—); 1.64 (m, 4H, OCOCH₂—CH₂—); 2.36 (t, 4H, OCOCH₂—CH₂—, J=7.4 Hz); 4.18 (multiplet, 5H, —CH₂—CH—CH₂—)

MS (MALDI-TOF): M+23=451 (M+Na⁺); M+39=467 (M+K⁺)

### Example 4

### Preparation of 1,2,3-triacylglycerols

### Example 4a

### Preparation of 1,2,3-tritetradecylthioacetylglycerol

Glycerol (1 g, 10.86 mmol) was dissolved in dichloromethane (200 ml). Dicyclohexylcarbodiimide (7.84 g, 38.01 mmol), dimethylaminopyridine (4.64 g, 38.01 mmol) and tetradecylthioacetic acid (example 1a) (9.40 g, 32.58 mmol) were then added. The mixture was stirred at room temperature. After 48 hours of reaction, the dicyclohexylurea precipitate was filtered, washed with dichloromethane and the filtrate was evaporated. The residue obtained was purified by silica gel chromatography (eluent:dichloromethane/cyclohexane 4:6). 1,2,3-tritetradecylthioacetylglycerol was obtained as a white powder.

Yield: 65%

Rf (dichloromethane/cyclohexane 7:3): 0.47

MP: 57° C.

IR: νCO ester 1738 and 1722 cm⁻¹

NMR (¹H, CDCl₃): 0.89 (t, 9H, —CH₃, J=6.5 Hz); 1.26 (multiplet, 66H, —CH₂—); 1.62 (m, 6H, —CH₂—CH₂—CH₂—S—); 2.63 (t, 6H, CH₂—CH₂—S—, J=7.3 Hz); 3.23 (s, 6H, S—CH₂—COO); 4.27 (dd, 2H, —CHaHb-CH—CHaHb-, J=12 Hz and J=6 Hz); 4.39 (dd, 2H, —CHaHb-CH—CHaHb-, J=12 Hz and J=4.3 Hz); 5.34 (m, 1H, —CHaHb-CH—CHaHb-)

MS (MALDI-TOF): M+23=925 (M+Na⁺); M+39=941 (M+K⁺)

### Example 4b

### Preparation of 1,2,3-tri-(4-dodecylthio)butanoylglycerol

This compound was obtained according to the procedure described hereinabove (example 4a) from 4-(dodecylthio)butanoic acid (example 1b) and glycerol.

Rf (dichloromethane/cyclohexane 7:3): 0.43

IR: νCO ester 1738 and 1727 cm⁻¹

NMR (¹H, CDCl₃): 0.84-0.92 (t, 9H, —CH₃, J=6.3 Hz); 1.22-1.44 (multiplet, 54H, —CH₂—); 1.50-1.64 (multiplet, 6H, —CH₂—CH₂—S—CH₂—CH₂—CH₂—COO); 1.83-1.97 (multiplet, 6H, —CH₂—S—CH₂—CH₂CH₂—COO); 2.42-2.59 (multiplet, 18H, —CH₂—CH₂—CH₂—S—CH₂—CH₂—CH₂—COO); 4.11-4.20 (dd, 2H, —CHaHb-CH—CHaHb-, J=12 Hz and J=5.9 Hz); 4.29-4.36 (dd, 2H, —CHaHb-CH—CHaHb-, J=12 Hz and J=4.5 Hz); 5.22-5.32 (m, 1H, —CHaHb-CH—CHaHb-)

MS (MALDI-TOF): M+23=925 (M+Na⁺); M+39=941 (M+K⁺)

### Example 4c

### Preparation of 1,2,3-tri-(6-decylthio)hexanoylglycerol

This compound was obtained according to the procedure described hereinabove (example 4a) from 6-(decylthio)hexanoic acid (example 1c) and glycerol.

Rf (dichloromethane/cyclohexane 7:3): 0.43

IR: νCO ester 1730 cm⁻¹

NMR (1H, CDCl₃): 0.85-0.92 (t, 9H, —CH₃, J=6.5 Hz); 1.21-1.50 (multiplet, 48H, —CH₂—); 1.51-1.72 (multiplet, 18H, —CH₂—CH₂—S—CH₂—CH₂—CH₂—CH₂—CH₂—COOH); 2.28-2.40 (multiplet, 6H, —CH₂—S—CH₂—CH₂—CH₂—CH₂—CH₂—COO); 2.45-2.57 (multiplet, 12H, —CH₂—S—CH₂—); 4.10-4.20 (dd, 2H, —CHaHb-CH—CHaHb-, J=12 Hz and J=6 Hz); 4.25-4.38 (dd, 2H, —CHaHb-CH—CHaHb-, J=12 Hz and J=4.3 Hz); 5.22-5.32 (m, 1H, —CHaHb-CH—CHaHb-)

MS (MALDI-TOF): M+23=925 (M+Na⁺); M+39=941 (M+K⁺)

### Example 4d

### Preparation of 1,2,3-tritetradecylsulfoxyacetylglycerol

This compound was obtained according to the procedure described hereinabove (example 4a) from tetradecylsulfoxyacetic acid (example 1e) and glycerol.

Rf (dichloromethane/cyclohexane 7:3): 0.33

IR: νCO ester 1730 cm⁻¹

NMR (1H, CDCl₃): 0.80-0.92 (t, 9H, —CH₃, J=6.4 Hz); 1.20-1.39 (multiplet, 60H, —CH₂—); 1.40-1.55 (multiplet, 6H, CH₂—); 1.70-1.90 (quint, 6H, —CH₂—CH₂—SO—); 2.82-2.89 (m, 6H, —CH₂—CH₂—SO—CH₂—COO—); 3.49-3.90 (m, 6H, —CH₂—SO—CH₂—COO); 4.10-4.30 (m, 2H, —CH₂—CH—CH₂—); 4.30-4.60 (m, 2H, —CH₂—CH—CH₂—); 5.45 (m, 1H, —CH₂—CH—CH₂—).

MS (MALDI-TOF): M+1=951; M+23=974 (M+Na⁺); M+39=990 (M+K⁺)

### Example 4e

### Preparation of 1,2,3-tri-(tetradecylsulfonyl)acetylglycerol

This compound was obtained according to the procedure described hereinabove (example 4a) from tetradecylsulfonylacetic acid (example 1g) and glycerol.

Rf (dichloromethane/ethyl acetate 9:1): 0.51

MP: 107.0 to 110.6° C.

IR: νCO ester 1769, 1754 and 1735 cm⁻¹; νSO 1120 cm⁻¹

NMR (¹H, CDCl₃): 0.87 (t, 9H, —CH₃, J=6.5 Hz); 1.19-1.35 (multiplet, 60H, —CH₂—); 1.44-1.49 (m, 6H, —CH₂—CH₂—CH₂—SO₂—); 1.81-1.92 (m, 6H, —CH₂—CH₂—SO₂—); 3.23 (t, 6H, —CH₂—CH₂—SO₂—CH₂—COO, J=7.5 Hz); 4.01 (s, 4H, —CH₂—SO₂—CH₂—COO); 4.03 (s, 2H, —CH₂—SO₂—CH₂—COO); 4.67 (m, 4H, —CH₂—CH—CH₂—); 5.49 (m, 1H, —CH₂—CH—CH₂—).

MS (MALDI-TOF): M+23=1021 (M+Na⁺); M+39=1037 (M+K⁺)

### Example 4f

### Preparation of 1,2,3-tri-tetradecylselenoacetylglycerol

This compound was obtained according to the procedure described hereinabove (example 4a) from tetradecylselenoacetic acid (example 1d) and glycerol.

Rf (dichloromethane/cyclohexane 7:3): 0.74

IR: νCO ester 1737 and 1721 cm⁻¹

NMR (1H, CDCl₃): 0.85-0.92 (t, 9H, —CH₃, J=6.2 Hz); 1.23-1.46 (multiplet, 66H, —CH₂—); 1.62-1.76 (multiplet, 6H, —CH₂—CH₂—CH₂—Se—); 2.72-2.79 (t, 6H, CH₂—CH₂—Se—, J=7.4 Hz); 3.15 (s, 6H, Se—CH₂—COO); 4.10-4.30 (m, 2H, —CH₂—CH—CH₂—); 4.30-4.60 (m, 2H, —CH₂—CH—CH₂—); 5.37 (m, 1H, —CH₂—CH—CH₂—).

### Example 4g

### Preparation of 1,3-dipalmitoyl-2-tetradecylthioacetylglycerol

1,3-dipalmitoylglycerol (example 3a) (5.64 g, 9.9 mmol, 1 eq), tetradecylthioacetic acid (example 1a) (5.74 g, 19.8 mmol, 2 eq), dicyclohexylcarbodiimide (4.1 g, 19.8 mmol, 2 eq) and dimethylaminopyridine (2.42 g, 19.8 mmol, 2 eq) were dissolved in dichloromethane. The reaction mixture was stirred at room temperature for 3 days. The dicyclohexylurea which formed was filtered and washed several times with dichloromethane. The filtrate was dried. The residual product was purified by silica gel chromatography (eluent:dichloromethane/cyclohexane 4:6).

Yield: 80%

Rf (dichloromethane/cyclohexane 7:3): 0.32

MP: 60-62° C.

IR: νCO ester 1744 and 1730 cm⁻¹

NMR (1H, CDCl₃): 0.86-0.91 (t, 9H, —CH₃, J=6.6 Hz); 1.10-1.45 (multiplet, 70H, —CH₂—); 1.57-1.64 (multiplet, 6H, —CH₂—CH₂—CH₂—S— and OCOCH₂—CH₂); 2.30-2.35 (t, 4H, OCOCH₂—CH₂—, J=7.4 Hz); 2.60-2.66 (t, 2H, CH₂—CH₂—S—, J=7.4 Hz); 3.23 (s, 2H, S—CH₂—COO); 4.14-4.21 (dd, 2H, —CHaHb-CH—CHaHb-, J=12 Hz and J=5.8 Hz); 4.30-4.36 (dd, 2H, —CHaHb-CH—CHaHb-, J=12 Hz and J=4 Hz); 5.26-5.33 (m, 1H, —CHaHb-CH—CHaHb-)

MS (MALDI-TOF): M+23=861 (M+Na⁺); M+39=877 (M+K⁺)

### Example 4h

### Preparation of 1,3-dilinoleyl-2-tetradecylthioacetylglycerol

This compound was obtained according to the procedure described hereinabove (example 4g) from 1,3-dilinoleylglycerol (example 3b) and tetradecylthioacetic acid (example 1a). The product was obtained as a colorless, viscous oil.

Yield: 56%

RF (dichloromethane/cyclohexane 7:3): 0.32

IR: νCO ester 1745 cm⁻¹

NMR (¹H, CDCl₃): 0.82-0.93 (t, 9H, —CH₃, J=6.6 Hz); 1.15-1.45 (multiplet, 50H, —CH₂—); 1.52-1.70 (multiplet, 6H, —CH₂—CH₂—CH₂—S— and OCOCH₂—CH₂); 1.93-2.14 (multiplet, 8H, —CH₂—CH═CH—CH₂—); 2.28-2.37 (t, 4H, OCOCH₂—CH₂—, J=7.5 Hz); 2.59-2.67 (t, 2H, CH₂—CH₂—S—, J=7.4 Hz); 2.70-2.83 (t, 4H, —CH₂—CH═CH—CH₂—CH═CH—CH₂—); 3.22 (s, 2H, S—CH₂—COO); 4.12-4.23 (dd, 2H, —CHaHb-CH—CHaHb-, J=12 Hz anf J=6.2 Hz); 4.28-4.37 (dd, 2H, —CHaHb-CH—CHaHb-, J=12 Hz and J=4 Hz); 5.24-5.45 (m, 1H, —CHaHb-CH—CHaHb-)

MS (MALDI-TOF): M+23=909 (M+Na⁺); M+39=925 (M+K⁺)

### Example 4i

### Preparation of 1,3-distearyl-2-tetradecylthioacetylglycerol

This compound was obtained according to the procedure described hereinabove (example 4g) from 1,3-distearylglycerol (compound 3c) and tetradecylthioacetic acid (compound 1a).

Yield: 41%

RF (dichloromethane): 0.32

IR: νCO ester 1744 and 1731 cm⁻¹

NMR (¹H, CDCl₃): 0.86-0.91 (t, 9H, —CH₃, J=6.6 Hz); 1.10-1.45 (multiplet, 78H, —CH₂—); 1.57-1.64 (multiplet, 6H, —CH₂—CH₂—CH₂—S— and OCOCH₂—CH₂); 2.29-2.35 (t, 4H, OCOCH₂—CH₂—, J=7.4 Hz); 2.60-2.66 (t, 2H, CH₂—CH₂—S—, J=7.4 Hz); 3.23 (s, 2H, S—CH₂—COOH); 4.14-4.21 (dd, 2H, —CHaHb-CH—CHaHb-, J=12 Hz and J=5.8 Hz); 4.30-4.36 (dd, 2H, —CHaHb-CH—CHaHb-, J=12 Hz and J=4 Hz); 5.26-5.32 (m, 1H, —CHaHb-CH—CHaHb-)

### Example 4j

### Preparation of 1,3-oleoyl-2-tetradecylthioacetylglycerol

This compound was obtained according to the procedure described hereinabove (example 4g) from 1,3-dioleoylglycerol (compound 3d) and tetradecylthioacetic acid (compound 1a). The product was obtained as a colorless, viscous oil.

Yield: 32%

RF (dichloromethane/cyclohexane 7:3): 0.50

IR: νCO ester 1746 cm⁻¹

NMR (1H, CDCl₃): 0.89 (t, 9H, —CH₃, J=6.4 Hz); 1.31 (multiplet, 66H, —CH₂—); 1.60 (multiplet, 6H, —CH₂—CH₂—CH₂—S— and OCOCH₂—CH₂); 2.02 (multiplet, 8H, —CH₂—CH═CH—CH₂—); 2.33 (t, 4H, OCOCH₂—CH₂—, J=7.3 Hz); 2.63 (t, 2H, CH₂—CH₂—S—, J=7.7 Hz); 3.23 (s, 2H, S—CH₂—COO); 4.18 (dd, 2H, —CHaHb-CH—CHaHb-, J=12.4 Hz and J=6.4 Hz); 4.33 (dd, 2H, —CHaHb-CH—CHaHb-, J=12.4 Hz and J=4.5 Hz); 5.33 (multiplet, 1H, —CHaHb-CH—CHaHb- and —CH₂—CH═CH—CH₂—)

MS (MALDI-TOF): M+23=913 (M+Na⁺); M+39=929 (M+K⁺)

### Example 4k

### Preparation of 1,3-ditetradecanoyl-2-tetradecylthioacetylglycerol

This compound was obtained according to the procedure described hereinabove (example 4g) from 1,3-ditetradecanoylglycerol (compound 3e) and tetradecylthioacetic acid (compound 1a).

Yield: 28%

RF (dichloromethane/cyclohexane 7:3): 0.30

MP: 60-62° C.

IR: νCO ester 1744 and 1730 cm⁻¹

NMR (¹H, CDCl₃): 0.87 (t, 9H, —CH₃, J=7.2 Hz); 1.27 (multiplet, 62H, —CH₂—); 1.60 (multiplet, 6H, —CH₂—CH₂—CH₂—S— and OCOCH₂—CH₂); 2.33 (t, 4H, OCOCH₂—CH₂—, J=7.7 Hz); 2.63 (t, 2H, CH₂—CH₂—S—, J=7.2 Hz); 3.23 (s, 2H, S—CH₂—COO); 4.18 (dd, 2H, —CHaHb-CH—CHaHb-, J=12 Hz and J=5.8 Hz); 4.33 (dd, 2H, —CHaHb-CH—CHaHb-, J=11.5 Hz and J=5.8 Hz); 5.30 (m, 1H, —CHaHb-CH—CHaHb-).

MS (MALDI-TOF): M+23=805 (M+Na⁺)

### Example 41

### Preparation of 1-palmitoyl-2,3-ditetradecylthioacetylglycerol

Glycerol 1-palmitate (example 2b) (4.804 g, 14 mmol) was dissolved in dichloromethane (300 ml). Dicyclohexylcarbodiimide (7.498 g, 36 mmol), dimethylaminopyridine (4.439 g, 0.036 mol) and tetradecylthioacetic acid (example 1a) (8.386 g, 29 mmol) were then added. The reaction mixture was stirred at room temperature for 48 hours. The dicyclohexylurea precipitate was filtered and washed with dichloromethane. The filtrate was dried. The residue was purified by silica gel chromatography (eluent:dichloromethane/cyclohexane 4:6) to give the desired compound as a white powder.

Yield: 42%

RF (dichloromethane/cyclohexane 7:3): 0.31

MP: 57-59° C.

IR: νCO ester 1736 and 1722 cm⁻¹

NMR (¹H, CDCl₃): 0.89 (t, 9H, —CH₃, J=6.6 Hz); 1.27 (multiplet, 68H, —CH₂—); 1.60 (multiplet, 6H, —CH₂—CH₂—CH₂—S— and OCOCH₂—CH₂); 2.33 (t, 2H, OCOCH₂—CH₂—, J=7 Hz); 2.63 (t, 4H, CH₂—CH₂—S—, J=8.9 Hz); 3.23 (s, 4H, S—CH₂—COO); 4.23 (m, 2H, —CHaHb-CH—CHaHb-); 4.37 (m, 2H, —CHaHb-CH—CHaHb); 5.31 (m, 1H, —CHaHb-CH—CHaHb-)

MS (MALDI-TOF): M+23=893 (M+Na⁺); M+39=909 (M+K⁺)

### Example 4m

### Preparation of 1-oleyl-3-palmitoyl-2-tetradecylthioacetylglycerol

1-oleyl-3-palmitoylglycerol (example 3g) (2 g, 3 mmol) was dissolved in dichloromethane (150 ml). Dicyclohexylcarbodiimide (1.040 g, 5 mmol), dimethylaminopyridine (0.616 g, 5 mmol) and tetradecylthioacetic acid (example 1a) (1.455 g, 5 mmol) were then added. The mixture was stirred at room temperature for 24 hours. The dicyclohexylurea precipitate was filtered, washed with dichloromethane and the filtrate was concentrated. The residue obtained was purified by silica gel chromatography (eluent: dichloromethane/cyclohexane 4:6) to give the desired compound as an oil.

Yield: 49%

RF (dichloromethane/cyclohexane 7:3): 0.45

MP <4° C.

IR: νCO ester 1742 cm⁻¹

NMR (1H, CDCl₃): 0.89 (t, 9H, —CH₃, J=6.5 Hz); 1.26 (multiplet, 66H, —CH₂—); 1.60 (multiplet, 6H, —CH₂—CH₂—CH₂—S— and OCOCH₂—CH₂); 2.03 (multiplet, 4H, —CH₂—CH═CH—CH₂—); 2.33 (t, 4H, OCOCH₂—CH₂—, J=7.4 Hz); 2.63 (t, 2H, CH₂—CH₂—S—, J=7.4 Hz); 3.23 (s, 2H, S—CH₂—COO); 4.18 (dd, 2H, —CHaHb-CH—CHaHb-, J=12.2 Hz and J=6.1 Hz); 4.33 (dd, 2H, —CHaHb-CH—CHaHb-, J=12.2 Hz and J=4.4 Hz); 5.32 (multiplet, 3H, —CHaHb-CH—CHaHb- and —CH₂—CH═CH—CH₂—)

MS (MALDI-TOF): M+23=887 (M+Na⁺); M+39=903 (M+K⁺)

### Example 4n

### Preparation of 1,3-dipalmitoyl-2-docosylthioacetylglycerol

This compound was obtained according to the procedure described hereinabove (example 4g) from 1,3-dipalmitoylglycerol (example 3a) and docosylthioacetic acid (example 1i).

Yield: 77%

RF (dichloromethane/cyclohexane 7:3): 0.32

IR: νCO ester 1745 and 1730 cm⁻¹

NMR (1H, CDCl₃): 0.86-0.91 (t, 9H, —CH₃, J=6.6 Hz); 1.10-1.45 (multiplet, 86H, —CH₂—); 1.57-1.64 (multiplet, 6H, —CH₂—CH₂CH₂—S— and OCOCH₂—CH₂); 2.29-2.34 (t, 4H, OCOCH₂—CH₂—, J=7.5 Hz); 2.60-2.66 (t, 2H, CH₂—CH₂—S—, J=7.4 Hz); 3.23 (s, 2H, S—CH₂—COO—); 4.13-4.22 (dd, 2H, —CHaHb-CH—CHaHb-, J=12 Hz and J=5.8 Hz); 4.30-4.36 (dd, 2H, —CHaHb-CH—CHaHb-, J=12 Hz and J=4 Hz); 5.27-5.34 (m, 1H, —CHaHb-CH—CHaHb-)

### Example 4o

### Preparation of 1,3-ditetradecylthioacetyl-2-palmitoylglycerol

This compound was obtained according to the procedure described hereinabove (example 4g) from 1,3-ditetradecylthioacetylglycerol (example 3f) and palmitic acid.

Yield: 36%

MP: 59-61° C.

RF (dichloromethane/cyclohexane 7:3): 0.35

IR: νCO ester 1740 cm⁻¹

NMR (¹H, CDCl₃): 0.89 (t, 9H, —CH₃, J=6.5 Hz); 1.26 (multiplet, 68H, —CH₂—); 1.55-1.65 (multiplet, 6H, —CH₂—CH₂—CH₂—S— and —OCOCH₂—CH₂); 2.34 (td, 2H, OCOCH₂—CH₂—, J=7.7 Hz and J=1.9 Hz); 2.63 (td, 4H, CH₂—CH₂—S—, J=7.3 Hz and J=1.9 Hz); 3.23 (s, 4H, S—CH₂—COO—); 3.68 (dd, 2H, —CHaHb-CH—CHaHb-, J=10.4 Hz and J=4.6 Hz); 4.36 (dd, 2H, —CHaHb-CH—CHaHb-, J=11.9 Hz and J=4.2 Hz); 5.31 (m, 1H, —CHaHb-CH—CHaHb-)

MS (MALDI-TOF): M+23=893 (M+Na⁺); M+39=909 (M+K⁺)

### Example 4p

### Preparation of 1,3-diacetyl-2-tetradecylthioacetylglycerol

This compound was obtained according to the procedure described hereinabove (example 4g) from 1,3-diacetylglycerol (example 3h) and tetradecylthioacetic acid (example 1a).

Yield: 10%

RF (ethyl acetate/cyclohexane 3:7): 0.47

MP <4° C.

IR: νCO ester 1748 cm⁻¹

NMR (1H, CDCl₃): 0.89 (t, 3H, —CH₃, J=6.9 Hz); 1.26 (multiplet, 20H, —CH₂—); 1.60 (multiplet, 4H, —CH₂—CH₂—CH₂—S—); 2.09 (s, 6H, —OCOCH₃); 2.64 (t, 2H, CH₂—CH₂—S—, J=7.4 Hz); 3.24 (s, 2H, S—CH₂—COO); 4.17 (dd, 2H, —CHaHb-CH—CHaHb-, J=12 Hz and J=5.8 Hz); 4.34 (dd, 2H, —CHaHb-CH—CHaHb-, J=12 Hz and J=4 Hz); 5.28 (m, 1H, —CHaHb-CH—CHaHb-)

MS (MALDI-TOF): M+23=469 (M+Na⁺); M+39=485 (M+K⁺)

### Example 4q

### Preparation of 1,3-dioctanoyl-2-tetradecylthioacetylglycerol

This compound was obtained according to the procedure described hereinabove (example 4g) from 1,3-dioctanoylglycerol (example 3i) and tetradecylthioacetic acid (example 1a).

Yield: 88%

RF (dichloromethane 10): 0.52

MP <4° C.

IR: νCO ester 1745 cm⁻¹

NMR (¹H, CDCl₃): 0.89 (t, 9H, —CH₃, J=7.0 Hz); 1.27 (multiplet, 38H, —CH₂—); 1.60 (multiplet, 6H, —CH₂—CH₂—CH₂—S— and OCOCH₂—CH₂); 2.32 (t, 4H, OCOCH₂—CH₂—, J=7.3 Hz); 2.63 (t, 2H, CH₂—CH₂—S—, J=7.3 Hz); 3.23 (s, 2H, S—CH₂—COO); 4.17 (dd, 2H, —CHaHb-CH—CHaHb-, J=11.9 Hz and J=5.8 Hz); 4.33 (dd, 2H, —CHaHb-CH—CHaHb-, J=11.9 Hz and J=4.3 Hz); 5.30 (m, 1H, —CHaHb-CH—CHaHb-)

MS (MALDI-TOF): M+23=637 (M+Na⁺); M+39=653 (M+K⁺)

### Example 4r

### Preparation of 1,3-diundecanoyl-2-tetradecylthioacetylglycerol

This compound was obtained according to the procedure described hereinabove (example 4g) from 1,3-diundecanoylglycerol (example 3j) and tetradecylthioacetic acid (example 1a).

Yield: 28%

RF (dichloromethane/cyclohexane 7:3): 0.16

IR: νCO ester 1738 and 1725 cm⁻¹

NMR (1H, CDCl₃): 0.89 (t, 9H, —CH₃, J=6.9 Hz); 1.26 (multiplet, 50H, —CH₂—); 1.62 (multiplet, 6H, —CH₂—CH₂—CH₂—S— and OCOCH₂—CH₂); 2.33 (t, 4H, OCOCH₂—CH₂—, J=7.7 Hz); 2.63 (t, 2H, CH₂—CH₂—S—, J=7.3 Hz); 3.23 (s, 2H, S—CH₂—COO); 4.20 (dd, 2H, —CHaHb-CH—CHaHb-, J=12.1 Hz and J=6.1 Hz); 4.35 (dd, 2H, —CHaHb-CH—CHaHb-, J=12.1 Hz and J=4.5 Hz); 5.29 (m, 1H, —CHaHb-CH—CHaHb-)

MS (MALDI-TOF): M+23=722 (M+Na⁺); M+39=738 (M+K⁺)

### Example 5

### Preparation of 2-aminoglycerol derivatives

### Example 5a

### Preparation of 2-tetradecylthioacetamidopropane-1,3-diol

Tetradecylthioacetic acid (example 1a) (2.878 g, 10 mmol) and 2-amino-1,3-propanediol (1 g, 11 mmol) were placed in a flask and heated at 190° C. for 1 hour. After cooling to room temperature, the medium was taken up in chloroform and washed with water. The organic phase was dried on magnesium sulfate, filtered then evaporated to form a solid ochre residue. This residue was stirred in diethyl ether for 12 hours. The product was isolated by filtration in the form of a white powder.

Yield: 6%

RF (dichloromethane/methanol 9:1): 0.60

MP: 95-97° C.

IR: νCO amide 1640 cm⁻¹

NMR (1H, CDCl₃): 0.84-0.93 (t, 3H, —CH₃, J=6.4 Hz); 1.21-1.45 (multiplet, 22H, —CH₂—); 1.54-1.72 (m, 2H, —CH₂—CH₂—CH₂—S—); 2.52-2.59 (t, 2H, CH₂—CH₂—S—, J=7.1 Hz); 2.63 (sl, 2H, OH); 3.27 (s, 2H, S—CH₂—COO); 3.77-3.96 (multiplet, 4H, —CH₂—CH—CH₂—); 3.97-4.04 (m, 1H, —CH₂—CH—CH₂—); 7.55 (d, 1H, —CONH—, J=6.7 Hz).

MS (MALDI-TOF): M+1=362; M+23=384 (M+Na⁺); M+39=400 (M+K⁺)

### Example 5b

### Preparation of 2-tetradecylthioacetamido-1,3-ditetradecylthioacetyloxypropane

2-tetradecylthioacetamidopropan-1,3-diol (example 5a) (1 g, 2.77 mmol) was dissolved in dichloromethane (180 ml). Dicyclohexycarbodiimide (1.427 g, 6.91 mmol), dimethylaminopyridine (0.845 g, 6.91 mmol) and tetradecylthioacetic acid (example 1a) (1.995 g, 6.91 mmol) were then added in that order. The reaction mixture was stirred at room temperature for 48 hours. The dicyclohexylurea precipitate was filtered and washed with dichloromethane and the filtrate was concentrated. The residue obtained was purified by silica gel chromatography (eluent:dichloromethane/cyclohexane 7:3). The desired compound was obtained as a white powder.

Yield: 66%

RF (dichloromethane): 0.18

MP: 82-84° C.

IR: νCO ester 1715 and 1730 cm⁻¹; νCO amide 1648 cm⁻¹

NMR (¹H, CDCl₃): 0.84-0.95 (t, 9H, —CH₃, J=6.6 Hz); 1.22-1.45 (multiplet, 66H, —CH₂—); 1.54-1.69 (multiplet, 6H, —CH₂—CH₂—CH₂—S—); 2.48-2.55 (t, 2H, CH₂—CH₂—S—CH₂—CONH—, J=7.5 Hz); 2.59-2.70 (t, 4H, CH₂—CH₂—S—CH₂—COO—, J=7.2 Hz); 3.24 (s, 6H, S—CH₂—CO—); 4.18-4.35 (multiplet, 4H, —CH₂—CH—CH₂—); 4.47-4.60 (m, 1H, —CH₂—CH—CH₂—); 7.23 (d, 1H, —CONH—, J=8.5 Hz).

MS (MALDI-TOF): M+23=924 (M+Na⁺)

### Example 6

### Preparation of 2-thioglycerol derivatives

### Example 6a

### Preparation of 2-(tetradecylthio)thiolacetic acid

### Preparation of S-triphenylmethyl 2-(tetradecylthio)thioacetate

Triphenylmethylthiol (9.58 g, 35 mmol) was dissolved in dichloromethane, and dicyclohexylcarbodiimide (7.15 g, 35 mmol), dimethylaminopyridine (4.24 g, 35 mmol) and tetradecylthioacetic acid (example 1a) (10 g, 35 mmol) were then added. The reaction mixture was stirred at room temperature for 24 hours. The dicyclohexylcarbodiimide was filtered and washed with dichloromethane. The filtrate was dried. The residue was purified by silica gel chromatography (eluent:dichloromethane/cyclohexane 1:9).

Yield: 30%

RF (dichloromethane/cyclohexane 2:8): 0.43

MP: 45-50° C.

IR: νCO ester 1689 cm⁻¹

NMR (1H, CDCl₃): 0.89 (t, 3H, —CH₃, J=6.4 Hz); 1.26 (multiplet, 22H, —CH₂—); 1.51-1.54 (m, 2H, —CH₂—CH₂—CH₂—S—); 2.47 (t, 2H, CH₂—CH₂—S—CH₂—COS—, J=7.1 Hz); 3.30 (s, 2H, S—CH₂—COS—); 7.23 (multiplet, 15H, aromatic H).

### Preparation of 2-(tetradecylthio)thiolacetic acid

S-triphenylmethyl 2-(tetradecylthio)thioacetate (4.715 g, 9 mmol) was added in the cold to a suspension of mercuric acetate (5.495 g, 17 mmol) in dichloromethane (150 ml). The reaction mixture was stirred for 18 hours. The mixture was filtered on Celite® and washed with hot dichloromethane. The filtrate was evaporated to give a powdery residue which was taken up in absolute ethanol and filtered. Concentration of the filtrate led to a yellow oil which was used without further purification.

RF (dichloromethane/methanol 9:1): 0.58

### Example 6b

### Preparation of 2-iodo-1,3-ditetradecylthioacetoxypropane

1,3-ditetradecylthioacetylglycerol (example 3f) (2 g, 3 mmol) was dissolved in toluene (180 ml), then imidazole (0.538 g, 8 mmol), triphenylphosphine (2.072 g, 8 mmol) and iodine (1.604 g, 6 mmol) were added. The reaction mixture was stirred at room temperature and the progress of the reaction was followed by thin-layer chromatography. After 20 hours of reaction, a solution saturated in sodium sulfite was added until complete blanching of the medium. The medium was allowed to settle and the aqueous phase was extracted with toluene. The organic phases were combined and washed with a saturated aqueous sodium chloride solution. The organic phase was dried on magnesium sulfate, filtrered and the solvent was evaporated. The resulting residue (4.4 g) was purified by chromatography on a Puriflash column (eluent:dichloromethane/cyclohexane 1:9 then 3:7).

Yield: 95%

RF (dichloromethane/cyclohexane 6:4): 0.62

MP: 51-53° C.

NMR (1H, CDCl₃): 0.89 (t, 6H, —CH₃, J=6.6 Hz); 1.27 (multiplet, 44H, —CH₂—); 1.63 (multiplet, 4H, —CH₂—CH₂—CH₂—S—); 2.66 (t, 4H, CH₂—CH₂S—CH₂—COO—, J=7.4 Hz); 3.26 (s, 4H, S—CH₂—CO—); 4.42 (multiplet, 5H—CH₂—CH—CH₂—)).

MS (MALDI-TOF): M+23=765 (M+Na⁺); 781 (M+K⁺)

### Example 6c

### Preparation of 1,3-ditetradecylthioacetoxy-2-(2-tetradecylthio)methylcarbonylthiopropane

2-iodo-1,3-ditetradecylthioacetoxypropane (example 6b) (200 mg, 0.27 mmol) and 2-(tetradecylthio)thiolacetic acid (example 6a) (82 mg, 0.27 mmol) were dissolved in distilled tetrahydrofuran (30 ml). The reaction mixture was cooled in an ice bath before adding soduim hydride (22 mg, 0.54 mmol). The mixture was stirred at room temperature. After 48 hours, the sodium hydride was hydrolyzed and the tetrahydrofuran was evaporated. The medium was extracted with ethyl acetate; the organic phase was washed with water, dried on magnesium sulfate, filtered and evaporated. The resulting oily yellow residue (164 mg) was purified by short silica gel chromatography (eluent:dichloromethane/cyclohexane 5:5) to give the desired compound as a yellow oil.

RF (dichloromethane/cyclohexane 5:5): 0.20

IR: νCO ester 1737 cm⁻¹

NMR (1H, CDCl₃): 0.87 (t, 9H, —CH₃, J=6.7 Hz); 1.26 (multiplet, 66H, —CH₂—); 1.56-1.63 (multiplet, 6H, —CH₂—CH₂—CH₂—S—); 2.19 (s, 2H, S—CH₂—COS—); 2.65 (t, 4H, CH₂—CH₂—S—CH₂—COO—, J=7.5 Hz); 2.87 (t, 2H, CH₂—CH₂—S—CH₂—COS—, J=4.6 Hz); 3.22-3.26 (m, 1H, —CH₂—CH—CH₂—); 3.27 (s, 4H, S—CH₂—COO—); 3.97-4.02 (m, 2H, —CHaHb-CH—CHaHb-); 4.46-4.51 (m, 2H, —CHaHb-CH—CHaHb-).

MS (MALDI-TOF): M+1=919 (M+H⁺)

### Example 7

### Method of Preparation of the Inventive Compounds for In Vitro Experiments

To perform the in vitro experiments described in the following examples, the inventive compounds were prepared in the form of an emulsion as described below.

An emulsion comprising the inventive compound and phosphatidylcholine (PC) was prepared as described by Spooner et al. (Spooner, Clark et al. 1988). The inventive compound was mixed with PC in a 4:1 (m/m) ratio in chloroform, the mixture was dried under nitrogen, then vacuum evaporated overnight; the resulting powder was taken up in 0.16 M KCl containing 0.01 M EDTA and the lipid particles were dispersed by ultrasound for 30 minutes at 37° C. The liposomes so formed were then separated by ultracentrifugation (XL 80 ultracentrifuge, Beckman Coulter, Villepinte, France) at 25,000 rpm for 45 minutes to recover liposomes having a size greater than 100 nm and close to that of chylomicrons. Liposomes composed only of PC were prepared concurrently to use as negative control.

The composition of the liposomes in the inventive compound was estimated by using the enzyme calorimetric triglyceride assay kit. The assay was carried out against a standard curve, prepared with the lipid calibrator CFAS, Ref. 759350 (Boehringer Mannheim GmbH, Germany). The standard curve covered concentrations ranging from 16 to 500 μg/ml. 100 μl of each sample dilution or calibration standard were deposited per well on a titration plate (96 wells). 200 μl of triglyceride reagents, ref. 701912 (Boehringer Mannheim GmbH, Germany) were then added to each well, and the entire plate was incubated at 37° C. for 30 minutes. Optical densities (OD) were read on a spectrophotometer at 492 nm. Triglyceride concentrations in each sample were calculated from the standard curve plotted as a linear function y=ax+b, where y represents OD and x represents triglyceride concentrations.

Liposomes containing the inventive compounds, prepared in this manner, were used for in vitro experiments described in examples 9,10 and 11.

### Example 8

### Evaluation of the Antioxidant Properties of the Inventive Compounds

A—Protection Against LDL Oxidation Induced by Copper or azobis(2-amidinopropane)dihydrochloride (MPH):

Oxidation of LDL is an important modification which plays a major role in the onset and development of atherosclerosis (Jurgens, Hoff et al. 1987). The following protocol allows demonstration of the antioxidant properties of compounds. Unless otherwise indicated, all reagents were from Sigma (St Quentin, France).

LDL were prepared as described in Lebeau et al. (Lebeau, Furman et al. 2000). The solutions of the test compounds were prepared at 10⁻² M in ethanol and diluted in PBS so that the final concentration ranged from 0.1 to 100 μM with a total ethanol concentration of 1% (V/V).

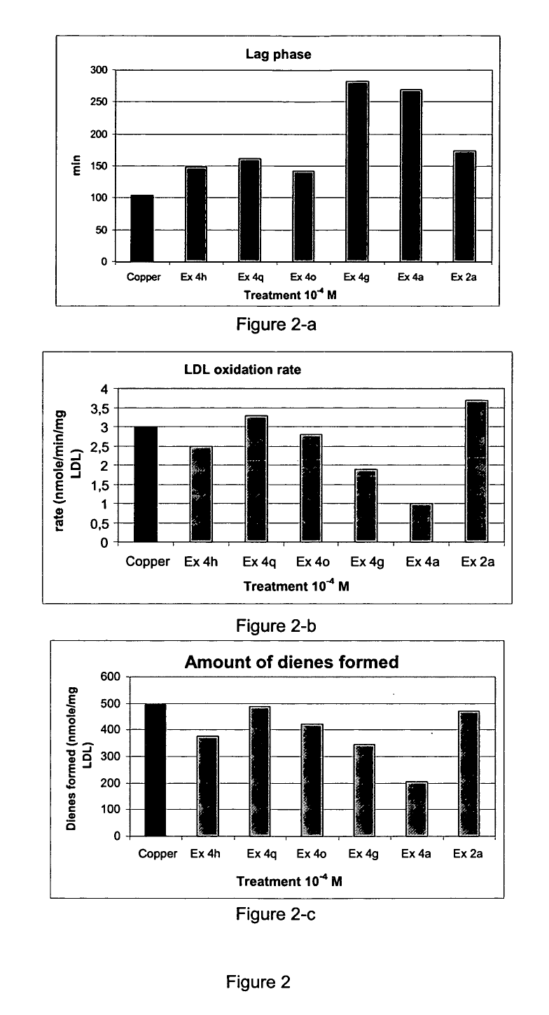

Before oxidation, EDTA was removed from the LDL preparation by dialysis. The oxidation reaction was then carried out at 30° C. by adding 100 μl of 16.6 μM CuSO₄ or 2 mM MPH to 800 μl of LDL (125 μg protein/ml) and 100 μl of a test compound solution. The formation of dienes, the species to be followed, was measured by the optical density at 234 nm in the samples treated with the compounds in the presence or absence of copper (or MPH). Optical density at 234 nm was measured every 10 minutes for 8 hours on a thermostated spectrophotometer (Kontron Uvikon 930). The analyses were carried out in triplicate. A compound was considered to have antioxidant activity when it shifted the lag phase latency relative to the control sample. The inventors demonstrate that the inventive compounds delay LDL oxidation (induced by copper), indicating that the inventive compounds possess intrinsic antioxidant activity. FIG. 2 presents an example of the results obtained with the inventive compounds.

FIG. 2a shows that incubation of LDL with the inventive compounds delayed conjugated diene formation. The lag phase was 104 minutes for copper alone as compared with a lag phase for conjugated diene formation that reached 282 minutes when LDL were incubated with inventive compound Ex 4g (inventive compound described in example 4g hereinabove) at 10⁻⁴ M. Inventive compound Ex 4a also increased the lag phase to 270 minutes. Said two compounds induced an increase in the lag phase of 170 and 160%, respectively. Compounds Ex 4h, 4q, 4o and 2a induced a 43, 54, 37 and 67% increase in the lag phase, respectively. This lag in the formation of conjugated dienes is characteristic of antioxidants. Inventive compounds Ex 4g and 4a were those with the most marked intrinsic antioxidant properties.

FIG. 2b shows that incubation of the inventive compounds with LDL in the presence of copper slowed the rate of conjugated diene formation. This rate was 3 nmol/min/mg of LDL with copper alone, and decreased to 1 nmol/min/mg of LDL with compound Ex 4a at 10⁻⁴ M, which corresponds to a 66% decrease in the oxidation rate. Inventive compounds Ex 4h and Ex 4g also slowed the LDL oxidation rate which in this case was 2.5 and 1.8 nmol/min/mg of LDL, respectively. Incubation of LDL with inventive compounds Ex 4q, 4o and 2a did not significantly alter the LDL oxidation rate.

Inventive compounds Ex 4a, 4g and 4h have intrinsic antioxidant properties and also promoted a slowing of the rate of copper-induced LDL oxidation.

FIG. 2c shows that incubation of LDL with copper led to the formation of 496 nmol of conjugated dienes per mg of LDL. Incubation with compound Ex 4a (10⁻⁴ M) led to a 60% decrease in the maximum amount of conjugated dienes formed. Compounds Ex 4g and 4h (10⁻⁴ M) also inhibited conjugated diene formation. Incubation of LDL with said compounds led to a respective 31 and 24% decrease in the maximum amount of conjugated dienes formed.

B—Evaluation of the Protection Conferred by the Inventive Compounds Against Lipid Peroxidation:

The inventive compounds which were tested are the compounds whose preparation is described in the hereinabove examples.

LDL oxidation was measured by the TBARS method.

According to the same principle as that described hereinabove, LDL were oxidized in the presence of CuSO₄ and lipid peroxidation was evaluated as follows:

TBARS were measured by a spectrophotometric method, lipid hydroperoxidation was measured by using lipid peroxide-dependent oxidation of iodide to iodine. The results are expressed as nmol of malondialdehyde (MDA) or as nmol hydroperoxide/mg protein.

The results obtained hereinabove by measuring the inhibition of conjugated diene formation, were confirmed by the experiments measuring LDL lipid peroxidation. The inventive compounds also afforded efficient protection of LDL against lipid peroxidation induced by copper (an oxidizing agent).

### Example 9

### Measurement of the Antioxidant Properties of the Inventive Compounds on Cell Cultures

A—Culture Protocol:

Neuronal, neuroblastoma (human) and PC12 cells (rat) were the cell lines used for this type of study. PC12 cells were prepared from a rat pheochromocytoma and have been characterized by Greene and Tischler (Greene and Tischler, 1976). These cells are commonly used in studies of neuron differentiation, signal transduction and neuronal death. PC12 cells were grown as previously described (Farinelli, Park et al. 1996) in complete RPMI medium (Invitrogen) supplemented with 10% horse serum and 5% fetal calf serum.

Primary cultures of endothelial and smooth muscle cells were also used. Cells were obtained from Promocell (Promocell GmBH, Heidelberg) and cultured according to the supplier's instructions.

The cells were treated with different doses of the compounds ranging from 5 to 300 μM for 24 hours. The cells were then recovered and the increase in expression of the target genes was evaluated by quantitative PCR.

B—mRNA Measurement:

mRNA was extracted from the cultured cells treated or not with the inventive compounds. Extraction was carried out with the reagents of the Absolutely RNA RT-PCR miniprep kit (Stratagene, France) as directed by the supplier. mRNA was then assayed by spectrometry and quantified by quantitative RT-PCR with a Light Cycler Fast Start DNA Master Sybr Green I kit (Roche) on a Light Cycler System (Roche, France). Primer pairs specific for the genes encoding the antioxidant enzymes superoxide dismutase (SOD), catalase and glutathione peroxidase (GPx) were used as probes. Primer pairs specific for the □-actin and cyclophilin genes were used as control probes.

An increase in mRNA expression of the antioxidant enzyme genes, measured by quantitative RT-PCR, was demonstrated in the different cell types used, when the cells were treated with the inventive compounds.

C—Control of Oxidative Stress:

Measurement of Oxidizing Species in the Cultured Cells:

The antioxidant properties of the compounds were also evaluated by means of a fluorescent tag the oxidation of which is followed by appearance of a fluorescence signal. The reduction in the intensity of the emitted fluorescence signal was determined in cells treated with the compounds in the following manner: PC12 cells cultured as described earlier (black 96-well plates, transparent bottom, Falcon) were incubated with increasing doses of H₂O₂ (0.25 mM-1 mM) in serum-free medium for 2 and 24 hours. After incubation, the medium was removed and the cells were incubated with 10 μM dichlorodihydrofluorescein diacetate solution (DCFDA, Molecular Probes, Eugene, USA) in PBS for 30 min at 37° C. in a 5% CO₂ atmosphere. The cells were then rinsed with PBS. The fluorescence emitted by the oxidation tag was measured on a fluorimeter (Tecan Ultra 384) at an excitation wavelength of 495 nm and an emission wavelength of 535 nm. The results are expressed as the percentage of protection relative to the oxidized control.

Fluorescence intensity was lower in the cells incubated with the inventive compounds than in untreated cells. These findings indicate that the inventive compounds promote inhibition of the production of oxidative species in cells subjected to oxidative stress. The previously described antioxidant properties are also effective at inducing antiradical protection in cultured cells.

D—Measurement of Lipid Peroxidation:

The different cell lines (cell models noted hereinabove) and the primary cell cultures were treated as described earlier. The cell supernatant was recovered after treatment and the cells were lysed and recovered for determination of protein concentration. Lipid peroxidation was detected as follows: lipid peroxidation was measured by using thiobarbituric acid (TBA) which reacts with lipid peroxidation of aldehydes such as malondialdehyde (MDA). After treatment, the cell supernatant was collected (900 μl) and 90 μl of butylated hydroxytoluene were added (Morliere, Moysan et al. 1991). One milliliter of 0.375% TBA solution in 0.25 M potassium carbonate containing 15% trichloroacetic acid was also added to the reaction medium. The mixture was heated at 80° C. for 15 min, cooled on ice and the organic phase was extracted with butanol. The organic phase was analyzed by spectrofluorimetry (λexc=515 nm and λem=550 nm) on a Shimazu 1501 spectrofluorimeter (Shimadzu Corporation, Kyoto, Japan). TBARS are expressed as MDA equivalents using tetra-ethoxypropane as standard. The results were normalized for protein concentration.

The decrease in lipid peroxidation observed in the cells treated with the inventive compounds confirms the previous results.

The inventive compounds advantageously exhibit intrinsic antioxidant properties allowing to slow and/or inhibit the effects of an oxidative stress. The inventors also show that the inventive compounds are capable of inducing the expression of genes encoding antioxidant enzymes. These particular features of the inventive compounds allow cells to more effectively fight against oxidative stress and therefore be protected against free radical-induced damage.

### Example 10

### Evaluation of PPAR Activation In Vitro by the Inventive Compounds

Nuclear receptors of the PPAR subfamily which are activated by two major pharmaceutical classes—fibrates and glitazones, widely used in the clinic for the treatment of dyslipidemias and diabetes—play an important role in lipid and glucose homeostasis. The following experimental data show that the inventive compounds activate PPARα in vitro.

PPAR activation was tested in vitro in RK13 fibroblast cell lines or in a hematocyte line HepG2 by measuring the transcriptional activity of chimeras composed of the DNA binding domain of the yeast gal4 transcription factor and the ligand binding domain of the different PPARs. The example below is given for HepG2 cells.

A—Culture Protocols:

HepG2 cells were from ECACC (Porton Down, UK) and were grown in DMEM medium supplemented with 10% (V/V) fetal calf serum, 100 U/ml penicillin (Gibco, Paisley, UK) and 2 mM L-glutamine (Gibco, Paisley, UK). The culture medium was changed every two days. Cells were kept at 37° C. in a humidified 95% air/5% CO₂ atmosphere.

B—Description of Plasmids Used for Transfection:

The plasmids pG5TkpGL3, pRL-CMV, pGal4-hPPARα, pGal4-hPPARγ and pGal4-f have been described by Raspe et al. (Raspe, Madsen et al. 1999). The pGal4-mPPARα and pGal4-hPPARβ constructs were obtained by cloning PCR-amplified DNA fragments corresponding to the DEF domains of the mouse PPARα and human PPARβ nuclear receptors, respectively, into the pGal4-f vector.

C—Transfection:

HepG2 cells were seeded in 24-well culture dishes at 5×10⁴ cells/well and transfected for 2 hours with the reporter plasmid pG5TkpGL3 (50 ng/well), the expression vectors pGal4-f, pGal4-mPPARα, pGal4-hPPARα, pGal4-hPPARγ, pGal4-hPPAβ (100 ng/well) and the transfection efficiency control vector pRL-CMV (1 ng/well) according to the previously described protocol (Raspe, Madsen et al. 1999), then incubated for 36 hours with the test compounds. At the end of the experiment, the cells were lysed (Gibco, Paisley, UK) and luciferase activity was determined with a Dual-Luciferase™ Reporter Assay System kit (Promega, Madison, Wis., USA) according to the supplier's instructions. The protein content of the cell extracts was then measured with the Bio-Rad Protein Assay (Bio-Rad, Munich, Germany) as directed by the supplier.

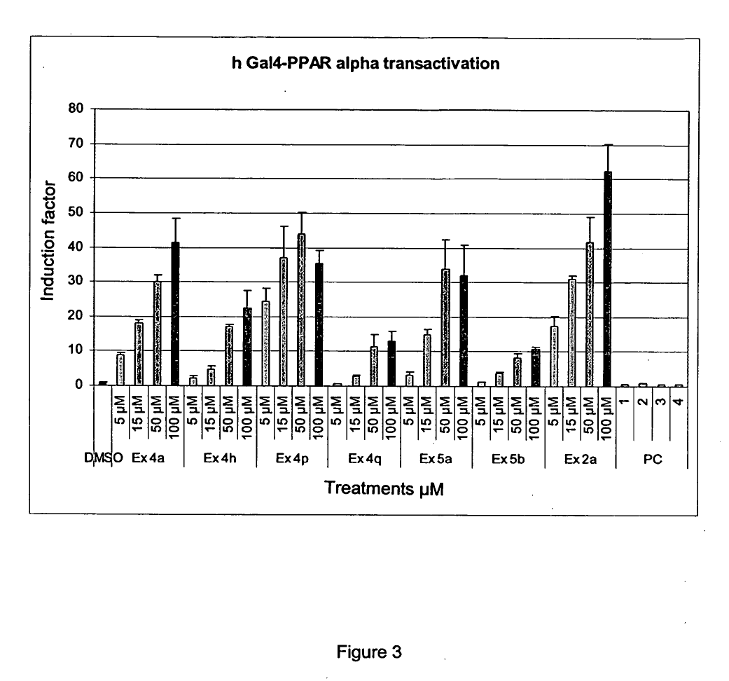

The inventors demonstrate an increase in luciferase activity in cells treated with the inventive compounds and transfected with the pGal4-hPPARα plasmid. Said induction of luciferase activity indicates that the inventive compounds are activators of PPAR□. FIG. 3 gives an example of the results obtained with the inventive compounds.

FIG. 3: HepG2 cells transfected with Gal4/PPARα plasmids were incubated with different concentrations of inventive compounds (5, 15, 50 and 100 μM) for 24 h and with different concentrations of the vehicle (PC). The results are expressed as the induction factor (luminescent signal relative to untreated cells) after the different treatments. The higher the induction factor the more potent the PPAR□ agonist activity. The results show that inventive compound Ex 2a produced a maximum 62-fold induction of the luminescent signal at 100 μM, 41 at 50 μM, 31 at 15 μM and 17 at 5 μM. Inventive compound Ex 4a also showed a dose-dependent increase in the induction factor of 41 at 100 μM, 30 at 50 μM, 18 at 15 μM and 9 at 5 μM. Inventive compound Ex 4p also induced an increase in the luminescent signal, revealing an activity on the PPARα nuclear receptor. The induction factors for compound Ex 4p were 35 at 100 μM, 44 at 50 μM, 36 at 15 μM and 24 at 5 μM. In contrast, when the cells were incubated with the vehicle (PC liposome), no significant induction was observed.

These results demonstrate that the inventive compounds tested exhibit significant PPAR□ ligand activity and therefore enable the transcriptional activation thereof.

### Example 11

### Evaluation of the Anti-Inflammatory Properties of the Inventive Compounds

An inflammatory response is observed in many neurological disorders, such as cerebral ischemias. Inflammation is also an important factor in neurodegeneration. In stroke, one of the first reactions of glial cells is to release cytokines and free radicals. This release of cytokines and free radicals results in an inflammatory response in the brain which can lead to neuronal death (Rothwell 1997).

Cell lines and primary cells were cultured as described hereinabove.

Lipopolysaccharide (LPS) bacterial endotoxin (Escherichia coli 0111:B4) (Sigma, France) was reconstituted in distilled water and stored at 4° C. Cells were treated with LPS 1 μg/ml for 24 hours. To avoid interference from other factors the culture medium was completely changed.

TNF-α is an important factor in the inflammatory response to stress (oxidative stress for example). To evaluate TNF-α secretion in response to stimulation by increasing doses of LPS, the culture medium of stimulated cells was removed and TNF-α was assayed with an ELISA-TNF-α kit (Immunotech, France). Samples were diluted 50-fold so as to be in the range of the standard curve (Chang, Hudson et al. 2000).

The anti-inflammatory property of the compounds was characterized as follows: the cell culture medium was completely changed and the cells were incubated with the test compounds for 2 hours, after which LPS was added to the culture medium at 1 μg/ml final concentration. After a 24-hour incubation, the cell supernatant was recovered and stored at 80° C. when not treated directly. Cells were lysed and protein was quantified with the Bio-Rad Protein Assay kit (Bio-Rad, Munich, Germany) according to the supplier's instructions.

The measurement of the decrease in TNF-α secretion induced by treatment with the test compounds is expressed as pg/ml/μg protein and as the percentage relative to the control. These results show that the inventive compounds have anti-inflammatory properties.

### Example 12

### Evaluation of the Neuroprotective Effects of the Inventive Compounds in a Cerebral Ischemia-Reperfusion Model

A—Prophylactic Model:

1—Treatment of Animals

1.1 Animals and Administration of the Compounds

Wistar rats weighing 200 to 350 g were used for this experiment.

Animals were maintained on a 12 hour light-dark cycle at a temperature of 20° C.±3° C. Water and food were available ad libitum. Food intake and weight gain were recorded.

Animals were treated by gavage with the inventive compounds (600 mg/kg/day) in suspension in the vehicle (0.5% carboxycellulose (CMC) and 0.1% Tween) or with the vehicle alone, for 14 days before ischemia induction by occlusion of the middle cerebral artery.

The carboxymethylcellulose used is a sodium salt of intermediate viscosity carboxymethylcellulose (Ref. C4888, Sigma-Aldrich, France). Tween used is Polyoxyethylenesorbitan Monooleate (Tween 80, Ref. P8074, Sigma-Aldrich, France).

1.2 Ischemia Induction-Reperfusion by Intraluminal Occlusion of the Middle Cerebral Artery:

Animals were anesthetized by intraperitoneal injection of 300 mg/kg chloral hydrate. A rectal probe was inserted and body temperature was maintained at 37° C.±0.5° C. Blood pressure was monitored throughout the experiment.

Under a surgical microscope, the right carotid artery was exposed by a median incision in the neck. The pterygopalatine artery was ligated at its origin and an arteriotomy was fashioned in the external carotid artery so as to insert a nylon monofilament, which was gently advanced to the common carotid artery and then into the internal carotid artery so as to occlude the origin of the middle cerebral artery. The filament was withdrawn one hour later to allow reperfusion.

2—Measurement of Brain Infarct Volume:

Twenty-four hours after reperfusion, animals previously treated or not with the inventive compounds were euthanized by pentobarbital overdose.

Brains were rapidly frozen and sliced. Sections were stained with cresyl violet. Unstained zones of the brain sections were considered to be damaged by the infarct. Areas (of the infarct and the two hemispheres) were measured and the volume of the infarct and the two hemispheres was calculated and the corrected infarct volume was determined by the following formula: (corrected infarct volume=infarct volume−(volume of right hemisphere−volume of left hemisphere)) to compensate for cerebral oedema.

Analysis of the brain sections from animals treated with the inventive compounds revealed a marked decrease in infarct volume as compared with untreated animals. When the inventive compounds were administered to the animals before the ischemia (prophylactic effect), they were capable of inducing neuroprotection.

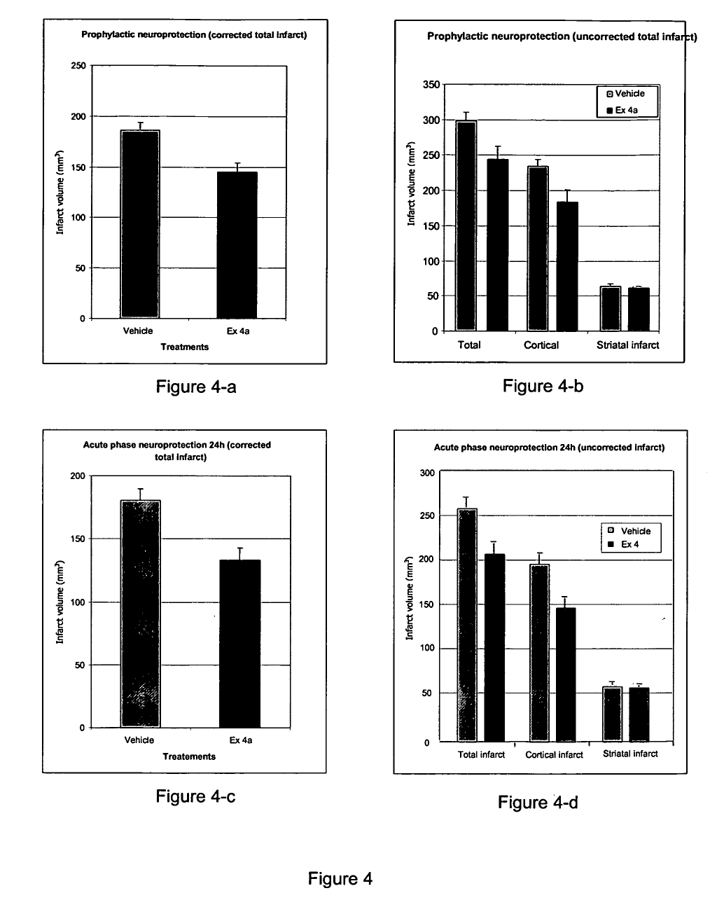

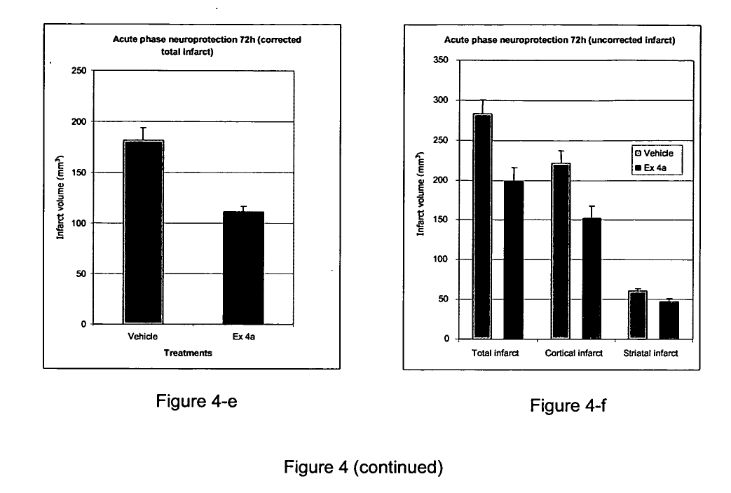

FIGS. 4a and 4b give an example of the results obtained with an inventive compound.

The results in FIG. 4a show that the corrected total infarct volume (post-ischemia lesion size) was 186 mm³. When animals were treated with compound Ex 4a (inventive compound described in example 4a) by the oral route, at 300 mg/kg/day bid, for 14 days before ischemia induction, lesion size decreased by 22% (145 mm³) relative to lesion size in control animals.

The results in FIG. 4b depicting uncorrected infarct volume indicate that the curative and neuroprotective feature of inventive compound Ex 4a observed for the total infarct is composed of a neuroprotective effect at the level of the cortical infarct (22% decrease in lesions) but with no effect at the level of the striatal infarct (no significant decrease in lesions).

3—Measurement of Antioxidant Enzyme Activity:

The rat brains were frozen, crushed and reduced to powder, then resuspended in saline solution. The different enzyme activities were then measured as described by the following authors: superoxide dismutase (Flohe and Offing 1984); glutathione peroxidase (Paglia and Valentine 1967); glutathione reductase (Spooner, Delides et al. 1981); glutathione-S-transferase (Habig and Jakoby 1981); catalase (Aebi 1984).

Said different enzyme activities were increased in brain preparations from animals treated with the inventive compounds.

B—Curative or Acute Phase Treatment Model:

1—Ischemia Induction/Reperfusion by Intraluminal Occlusion of the Middle Cerebral Artery:

Animals such as those described previously were used for this experiment. Animals were anesthetized by intraperitoneal injection of 300 mg/kg chloral hydrate. A rectal probe was inserted and body temperature was maintained at 37° C.±0.5° C. Blood pressure was monitored throughout the experiment.

Under a surgical microscope, the right carotid artery was exposed by a median incision in the neck. The pterygopalatine artery was ligated at its origin and an arteriotomy was fashioned in the external carotid artery so as to insert a nylon monofilament, which was gently advanced to the common carotid artery and then into the internal carotid artery so as to occlude the origin of the middle cerebral artery. The filament was withdrawn one hour later to allow reperfusion.

2—Treatment of Animals:

Animals first subjected to ischemia-reperfusion were treated with the inventive compounds by the oral route (such as previously described in CMC+Tween vehicle) one or more times after reperfusion (600 mg/kg/day or 300 mg/kg/day bid).

3—Measurement of Brain Infarct Volume:

24, 48 or 72 hours after reperfusion, animals previously treated or not with the compounds were euthanized by pentobarbital overdose.

Brains were rapidly frozen and sliced. Sections were stained with cresyl violet. Unstained zones of the brain sections were considered to be damaged by the infarct. Areas (of the infarct and the two hemispheres) were measured, the volume of the infarct and the two hemispheres was calculated and the corrected infarct volume was determined by the following formula: (corrected infarct volume=infarct volume−(volume of right hemisphere−volume of left hemisphere)) to compensate for cerebral oedema.

In the case of curative treatment (treatment of the acute phase), animals treated with the inventive compounds had less brain damage than untreated animals. In fact, the infarct volume was smaller when the inventive compounds were administered one or more times after ischemia-reperfusion. FIGS. 4c to 4f give an example of the results obtained with an inventive compound.

The results in FIG. 4c show that animals treated with inventive compound Ex 4a (600 mg/kg/day) for 24 hours after ischemia developed lesions that were 27% smaller than control animals (infarct volume 132 mm³ for treated animals versus 180 mm³ for controls).

The results in FIG. 4d depicting uncorrected infarct volume indicate that the curative and neuroprotective feature of inventive compound Ex 4a observed for the total infarct is composed of a neuroprotective effect at the level of the cortical infarct (25% decrease in lesions) but with no effect at the level of the striatal infarct (no significant decrease in lesions).

The results in FIG. 4e show that animals treated with inventive compound Ex 4a (600 mg/kg/day) for 72 hours after ischemia developed lesions that were 40% smaller than control animals (corrected infarct volume 110 mm³ for treated animals versus 180 mm³ for controls).

The results in FIG. 4f depicting uncorrected infarct volume indicate that the curative and neuroprotective feature of inventive compound Ex 4a observed for the total infarct is composed of a neuroprotective effect at the level of the cortical infarct (32% decrease in lesions) but also at the level of the striatal infarct (23% decrease in lesions).

The use of the compounds according to the invention in different experimental models shows that said compounds have intrinsic antioxidant activity, are capable of delaying and reducing the effects of an oxidative stress, and furthermore also induce the expression of genes coding for antioxidant enzymes, which together with their antioxidant property reinforces the protection against free radicals. In addition, the inventive compounds exhibit anti-inflammatory activity and are capable of activating the PPAR [symbol] nuclear receptor

Finally, use of the inventive compounds in an animal ischemia-reperfusion model revealed the beneficial neuroprotective effect of both preventive and curative treatment.

### BIBLIOGRAPHIC REFERENCES

Adams, H. P., Jr. (2002). “Emergent use of anticoagulation for treatment of patients with ischemic stroke.” Stroke 33(3): 856-61.
Aebi, H. (1984). “Catalase in vitro.” Methods Enzymol 105: 121-6.
Aveta, R., A. Brandt, et al. (1986). “Syntheses of 2-mercapto- and 2-alkylthio-1,3-propanediol derivatives.” Gazz Chim Ital 116(11): 649-652.
Bodai, Novak, et al. (1999). “Synthesis and lipase-catalyzed enantiope selective acetylation of 2-benzoyloxy-1,3-propanediol.” Synlett 6: 759-761.
Bordet, R., D. Deplanque, et al. (2000). “Increase in endogenous brain superoxide dismutase as a potential mechanism of lipopolysaccharide-induced brain ischemic tolerance.” J Cereb Blood Flow Metab 20(8): 1190-6.
Chang, R. C., P. Hudson, et al. (2000). “Influence of neurons on lipopolysaccharide-stimulated production of nitric oxide and tumor necrosis factor-alpha by cultured glia.” Brain Res 853(2): 236-44.
Clark, R. B. (2002). “The role of PPARs in inflammation and immunity.” J Leukoc Biol 71(3): 388-400.
Daniher, A. and J. Bashkin (1998). “Precise control of RNA cleavage by ribozyme mimics.” Chem Commun 10: 1077-1078.
Daubert, Spiegl, et al. (1943). “Unsaturated synthetic glycerides. II. Unsymmetrical dioleo-monosaturated triglycerides.” J Am Chem Soc 65: 2145.
Dimagl, U., C. Iadecola, et al. (1999). “Pathobiology of ischaemic stroke: an integrated view.” Trends Neurosci 22(9): 391-7.
Farinelli, S. E., D. S. Park, et al. (1996). “Nitric oxide delays the death of trophic factor-deprived PC12 cells and sympathetic neurons by a cGMP-mediated mechanism.” J Neurosci 16(7): 2325-34.
Feuge, R. O., A. T. Gros, et al. (1953). “Modification of vegetable oils. XIV. Properties of aceto-oleins.” J Am Oil Chem Soc 30: 320-325.
Feuge, R. O. and N. V. Lovegren (1956). “Dilatometric properties of some butyropalmitins, butyrostearins, and acetopalmitins.” J Am Oil Chem Soc 33: 367-372.
Flohe, L. and F. Otting (1984). “Superoxide dismutase assays.” Methods Enzmmol 105: 93-104.
Fruchart, J. C., B. Staels, et al. (2001). “PPARS, metabolic disease and atherosclerosis.” Pharmacol Res 44(5): 345-52.
Gaffney, P. R. J. and C. B. Reese (1997). “Preparation of 2-O-arachidonoyl-1-O-stearoyl-sn-glycerol and other di-O-acyl glycerol derivatives.” Tetrahedron Lett 38(14): 2539-2542.
Gangadhar, A., R. Subbarao, et al. (1989). “A facile synthesis of 1(3)-acylglycerols.” Synth. Commun. 19: 2505-2514.
Gilgun-Sherki, Y., E. Melamed, et al. (2001). “Oxidative stress induced-neurodegenerative diseases: the need for antioxidants that penetrate the blood brain barrier.” Neuropharmacology 40(8): 959-75.
Greene, L. A. and A. S. Tischler (1976). “Establishment of a noradrenergic clonal line of rat adrenal pheochromocytoma cells which respond to nerve growth factor.” Proc Natl Acad Sci USA 73(7): 2424-8.
Gronowitz, S., B. Herslöf, et al. (1978). “Syntheses and chroptical properties of some derivatives of 1-thioglycerol.” Chem Phys Lipids 22: 307-320.
Habig, W. H. and W. B. Jakoby (1981). “Assays for differentiation of glutathione S-transferases.” Methods Enzmmol 77: 398-405.
Han, S. Y., S. H. Cho, et al. (1999). “Monoacetyldiglycerides as new Ca2+ mobilizing agents in rat pancreatic acinar cells.” Bioorg Med Chem Left 9(1): 59-64.
Harada, H., T. Morie, et al. (1996). “An efficient synthesis of 6-substituted aminohexahydro-1H-1,4-diazepines from 2-substituted aminopropenals.” Chem Pharm Bull 44(12): 2205-2212.
Jurgens, G., H. F. Hoff, et al. (1987). “Modification of human serum low density lipoprotein by oxidation—characterization and pathophysiological implications.” Chem Phys Lipids 45(2-4): 315-36.
Kainu, T., A. C. Wikstrom, et al. (1994). “Localization of the peroxisome proliferator-activated receptor in the brain.” Neuroreport 5(18): 2481-5.
Katoch, R., G. K. Trivedi, et al. (1999). “1-[2-Hydroxy-3-octadecan-1′-oate]propyl-2″,2″,5″,5″-tetramethylpyrolidine-N-oxyl-3″-carboxylate as a potential spin probe for membrane structure studies.” Bioorg Med Chem 7(12): 2753-8.
Khanolkar, A. D., V. Abadji, et al. (1996). “Head group analogs of arachidonylethanolamide, the endogenous cannabinoid ligand.” J Med Chem 39(22): 4515-9.
Kurfürst, R., V. Roig, et al. (1993). “Oligo-a-deoxyribonucleotides with a modified nucleic base and covalently linked to reactive agents.” Tetrahedron 49(32): 6975-6990.
Lebeau, J., C. Furman, et al. (2000). “Antioxidant properties of di-tert-butylhydroxylated flavonoids.” Free Radic Biol Med 29(9): 900-12.
Lutsep, H. L. and W. M. Clark (2001). “Current status of neuroprotective agents in the treatment of acute ischemic stroke.” Curr Neurol Neurosci Rep 1(1): 13-8.
Mates, J. M., C. Perez-Gomez, et al. (1999). “Antioxidant enzymes and human diseases.” Clin Biochem 32(8): 595-603.
Morliere, P., A. Moysan, et al. (1991). “UVA-induced lipid peroxidation in cultured human fibroblasts.” Biochim Biophys Acta 1084(3): 261-8.
Nandagopal, K., T. M. Dawson, et al. (2001). “Critical role for nitric oxide signaling in cardiac and neuronal ischemic preconditioning and tolerance.” J Pharmacol ExP Ther 297(2): 474-8.
Paglia, D. E. and W. N. Valentine (1967). “Studies on the quantitative and qualitative characterization of erythrocyte glutathione peroxidase.” J Lab Clin Med 70(1):158-69.
Paris, G. Y., D. L. Garmaise, et al. (1980). “Glycerides as prodrugs. 3. Synthesis and antiinflammatory activity of [1-(p-chlorobenzoyl)-5-methoxy-2-methylindole-3-acetyl]glycerides (indomethacin glycerides).” J Med Chem 23(1): 9-13.
Putnam, W. and J. Bashkin (2000). “De novo synthesis of artificial ribonucleases with benign metal catalysts.” Chem Commun 9: 767-768.
Raspe, E., L. Madsen, et al. (1999). “Modulation of rat liver apolipoprotein gene expression and serum lipid levels by tetradecylthioacetic acid (TTA) via PPARalpha activation.” J Lipid Res 40(11): 2099-110.
Robinson, N. (1960). “Micellar size and surface activity of some C18 a-monoglycerides in benzene.” J Pharm Pharmacol 12: 685-689.
Rothwell, N. J. (1997). “Cytokines and acute neurodegeneration.” Mol Psychiatry 2(2): 120-1.
Scriba, G. K. (1993). “Phenyloin-lipid conjugates as potential prodrugs of phenyloin.” Arch Pharm (Weinheim) 326(8): 477-81.
Seltzman, Fleming, et al. (2000). “Facile synthesis and stabilization of 2-arachidonylglycerol via its 1,3-phenylboronate ester.” Tetrahedron Lett 41(19): 3589-3592.
Shaban, M. (1977). “Determination of the position of linkage of 2-acetamido-2-deoxy-D-galactose and 2-acetamido-2-deoxy-D-glucose residues in oligosaccharides and glycoproteins. Synthesis of 2-acetamido-2-deoxy-D-xylitol and 2-acetamido-2-deoxy-L-threitol.” Carbohydr Res 59: 213-233.
Smith, K. J., E. Dipreta, et al. (2001). “Peroxisomes in dermatology. Part II.” J Cutan Med Surg 5(4): 315-22.
Spooner, P. J., S. B. Clark, et al. (1988). “The ionization and distribution behavior of oleic acid in chylomicrons and chylomicron-like emulsion particles and the influence of serum albumin.” J Biol Chem 263(3): 1444-53.
Spooner, R. J., A. Delides, et al. (1981). “Heat stability and kinetic properties of human serum glutathione reductase activity in various disease states.” Biochem Med 26(2): 239-48.
Strawn, L. M., R. E. Martell, et al. (1989). “Iodoaryl analogues of dioctanoylglycerol and 1-oleoyl-2-acetylglycerol as probes for protein kinase C.” J Med Chem 32(9): 2104-10.
Strawn, L. M., R. E. Martell, et al. (1989). “Synthesis and evaluation of iodinated analogues of diacylglycerols as potential probes for protein kinase C.” J Med Chem 32(3): 643-8.
Terradas (1993). “Marked dependence of enzyme prochiral selectivity on the solvent.” J Amer Chem Soc 115: 390-396.

1-24. (canceled)

25. A method for treating a cerebrovascular pathology, comprising administering to a subject in need of such treatment, an effective dose of a compound represented by the following general formula (I) 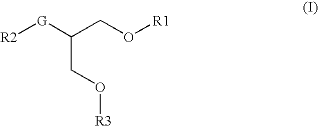
in which
G represents an oxygen atom, a sulfur atom or a N—R4 group,
R4 is a hydrogen atom or a linear or branched alkyl group, saturated or not, optionally substituted, containing from 1 to 5 carbon atoms,
R1, R2 and R3, which are the same or different, represent a hydrogen atom, a CO—R group or a group corresponding to the formula CO—(CH₂)₂ₙ₊₁—X—R′, at least one of the groups R1, R2 and R3 is a group corresponding to the formula CO—(CH₂)₂ₙ₊₁—X—R′,
R is a linear or branched alkyl group, saturated or not, optionally substituted, the main chain of which contains from 1 to 25 carbon atoms,
X is a sulfur atom, a selenium atom, a SO group or a SO₂ group,
n is a whole number comprised between 0 and 11,
R′ is a linear or branched alkyl group, saturated or not, optionally substituted, the main chain of which contains from 2 to 23, preferably 10 to 23, carbon atoms and possibly one or more heterogroups selected in the group consisting of an oxygen atom, a sulfur atom, a selenium atom, a SO group and a SO₂ group.

26. The method according to claim 25, in which the cerebrovascular pathology is cerebral ischemia or hemorrhagic stroke.

27. The method according to claim 25, wherein R group or groups, which are the same or different, represent a linear or branched alkyl group, saturated or unsaturated, substituted or not, the main chain of which contains from 1 to 20 carbon atoms, preferably from 7 to 17 carbon atoms, even more preferably 14 to 17 carbon atoms.

28. The method according to claim 25, wherein R′ group or groups, which are the same or different, represent a linear or branched alkyl group, saturated or unsaturated, substituted or not, the main chain of which contains from 12 to 23 carbon atoms, even more preferably from 13 to 20 carbon atoms, advantageously from 14 to 17 carbon atoms.

29. The method according to claim 25, wherein R group or groups, which are the same or different, are selected in the group consisting of C₇H₁₅, C₁₀H₂₁, C₁₁H₂₃, C₁₂H₂₅, C₁₃H₂₇, C₁₄H₂₉, C₁₆H₃₃, C₁₇H₃₅, C₁₅H₃₁, C₁₄H₂₇, C₁₄H₂₅, C₁₅H₂₉, C₁₇H₂₉, C₁₇H₃₁, C₁₇H₃₃, C₁₉H₂₉, C₁₉H₃₁, C₂₁H₃₁, C₂₁H₃₅, C₂₁H₃₇, C₂₁H₃₉, C₂₃H₄₅, the alkyl chains of eicosapentanoic acid (EPA) C_{20:5} (5, 8, 11, 14, 17) and docosahexanoic acid (DHA) C_{22:6} (4, 7,10, 13, 16, 19), (CH₂)_{n′}—CH(CH₃)C₂H₅, (CH═C(CH₃)(CH₂)₂)_{n″}—CH═C(CH₃)₂ and (CH₂)₂ₓ₊₁—C(CH₃)₂—(CH₂)_{n′″}—CH₃, x being a whole number equal to or comprised between 1 and 11, n′ being a whole number equal to or comprised between 1 and 22, n″ being a whole number equal to or comprised between 1 and 5, n′″ being a whole number equal to or comprised between 0 and 22 and (2x+n′″) being less than or equal to 22 preferably less than or equal to 20.

30. The method according to claim 25, wherein R′ group or groups, which are the same or different, are selected in the group consisting of C₇H₁₅, C₁₀H₂₁, C₁₁H₂₃, C₁₂H₂₅, C₁₃H₂₇, C₁₄H₂₉, C₁₆H₃₃, C₁₇H₃₅, C₁₅H₃₁, C_{20:5}(5, 8, 11, 14, 17), C_{22:6}(4, 7, 10, 13, 16, 19), C₁₄H₂₇, C₁₄H₂₅, C₁₅H₂₉, C₁₇H₂₉, C₁₇H₃₁, C₁₇H₃₃, C₁₉H₂₉, C₁₉H₃₁, C₂₁H₃₁, C₂₁H₃₅, C₂₁H₃₇, C₂₁H₃₉, C₂₃H₄₅, (CH₂)_{n′}—CH(CH₃)C₂H₅, (CH═C(CH₃)(CH₂)₂)_{n″}—CH═C(CH₃)₂ and (CH₂)₂ₓ₊₁—C(CH₃)₂—(CH₂)_{n′″}—CH₃, x being a whole number equal to or comprised between 1 and 11, n′ being a whole number equal to or comprised between 1 and 22, n″ being a whole number equal to or comprised between 1 and 5, n′″ being a whole number equal to or comprised between 0 and 22 and (2x+n′″) being less than or equal to 22, preferably less than or equal to 20.

31. The method according to claim 25, wherein R group or groups, which are the same or different, represent a lower alkyl group containing from 1 to 6 carbon atoms.

32. The method according to claim 25, wherein R′ group or groups, which are the same or different, are saturated and linear alkyl groups containing 14 carbon atoms.

33. The method according to claim 25, wherein the alkyl groups R or R′ are substituted by one or more substituents, which are the same or different, selected in the group consisting of a halogen atom (iodine, chlorine, fluorine, bromine) and a OH, ═O, NO₂, NH₂, CN, CH₂—OH, O—CH₃, CH₂OCH₃, CF₃ and COOZ group in which Z is a hydrogen atom or an alkyl group containing from 1 to 6 carbon atoms.

34. The method according to claim 25, wherein X is a sulfur or selenium atom, preferably a sulfur atom.

35. The method according to claim 25, wherein the group G represents an oxygen atom or a N—R4 group and, when G is N—R4, R4 preferably represents a hydrogen atom or a methyl group.

36. The method according to claim 25, wherein, in the group CO—(CH₂)₂ₙ₊₁—X—R′, n is comprised between 0 and 3, more specifically comprised between 0 and 2, and in particular is equal to 0.

37. The method according to claim 25, wherein at least one of the groups R1, R2 and R3 represents a CO—(CH₂)₂ₙ₊₁—X—R′ group in which X represents a selenium atom or preferably a sulfur atom and/or R′ is a saturated and linear alkyl group containing from 13 to 17 carbon atoms, preferably from 14 to 16, even more preferably 14 carbon atoms.

38. The method according to claim 25, wherein R2 is a group corresponding to the formula CO—(CH₂)₂ₙ₊₁—X—R′, preferably in which X represents a selenium atom or preferably a sulfur atom and/or R′ is a saturated and linear alkyl group containing from 13 to 17 carbon atoms, more preferably in which n is equal to 0, in particular a group having the formula CO—CH₂—S—C₁₄H₂₉.

39. The method according to claim 25, wherein R2 is a group corresponding to the formula CO—(CH₂)₂ₙ₊₁—X—R′, preferably in which X represents a selenium atom or preferably a sulfur atom and/or R′ is a saturated and linear alkyl group containing from 13 to 17 carbon atoms, more preferably in which n is equal to 0, in particular a group having the formula CO—CH₂—S—C₁₄H₂₉, and R1 and R3, which are the same or different, represent a hydrogen atom or a CO—R group.

40. The method according to claim 25, wherein R2 is a group corresponding to the formula CO—(CH₂)₂ₙ₊₁—X—R′, preferably in which X represents a selenium atom or preferably a sulfur atom and/or R′ is a saturated and linear alkyl group containing from 13 to 17 carbon atoms, more preferably in which n is equal to 0, in particular a group having the formula CO—CH₂—S—C₁₄H₂₉, and R1 and R3, which are the same or different, represent a CO—R group.

41. The method according to claim 25, wherein two of the groups R1, R2 and R3 are CO—(CH₂)₂ₙ₊₁—X—R′ groups, which are the same or different, preferably in which X represents a selenium atom or preferably a sulfur atom and/or R′ is a saturated and linear alkyl group containing from 13 to 17 carbon atoms, more preferably in which n is equal to 0, in particular CO—CH₂—S—C₁₄H₂₉ groups.

42. The method according to claim 25, wherein R1, R2 and R3, which are the same or different, preferably the same, are CO—(CH₂)₂ₙ₊₁—X—R′ groups, preferably in which X represents a selenium atom or preferably a sulfur atom and/or R′ is a saturated and linear alkyl group containing from 13 to 17 carbon atoms, more preferably in which n is comprised between 0 and 3, and in particular equal to 0.

43. The method according to claim 25, wherein R1, R2 and R3 represent CO—CH₂—S—C₁₄H₂₉ groups.

44. The method according to claim 25, wherein one or two of the substituents R1, R2 or R3 is a COCH₃ group.

45. The method according to claim 25, wherein group G represents a sulfur atom.

46. A compound represented by formula (I) such as defined in claim 25, selected in the group consisting of:
1,3-ditetradecylthioacetyl-2-palmitoylglycerol;
1,3-diacetyl-2-tetradecylthioacetylglycerol;
1,3-dioctanoyl-2-tetradecylthioacetylglycerol;
1,3-diundecanoyl-2-tetradecylthioacetylglycerol; and
1,3-ditetradecylthioacetoxy-2-(2-tetradecylthio)methylcarbonylthio-propane.

47. A pharmaceutical composition, comprising, in a pharmaceutically acceptable vehicle, at least one compound represented by general formula (I) identified in claim 46.

48. A pharmaceutical composition, comprising, in a pharmaceutically acceptable vehicle, at least one compound represented by general formula (I) identified in claim 46, for the treatment of cerebrovascular pathologies and more particularly cerebral ischemia or stroke.
